(19) United States
(12) Patent Application Publication
(10) Pub. No.: US 2024/0018217 A1
(43) Pub. Date: Jan. 18, 2024

(54) Fully Human Monoclonal Antibodies that Broadly Neutralize SARS-COV-2

(71) Applicant: Oklahoma Medical Research Foundation, Oklahoma City, OK (US)

(72) Inventor: Kenneth Smith, Norman, OK (US)

(21) Appl. No.: 18/344,680

(22) Filed: Jun. 29, 2023

Related U.S. Application Data
(60) Provisional application No. 63/356,682, filed on Jun. 29, 2022.

(51) Int. Cl. C07K 16/10; A61K 45/06; A61K 39/42; A61K 49/00; A61K 51/10; A61K 47/69; A61P 31/14; G01N 33/569

(52) U.S. Cl. CPC C07K 16/1003; A61K 45/06; A61K 39/42; A61K 49/0058; A61K 51/1006; A61K 47/6929; A61K 47/6911; A61P 31/14; G01N 33/56983; G01N 2333/165; G01N 2469/10

(57) ABSTRACT

The present invention includes antibodies and antigen-binding fragments thereof that specifically bind a SARS-CoV-2 antigen and are in certain embodiments capable of neutralizing a SARS-CoV-2 infection. Polynucleotides encoding an antibody or an antigen-binding fragment, vectors and host cells that comprise a polynucleotide and pharmaceutical compositions are also included in this invention. Also described herein are methods of using the presently disclosed antibodies, antigen-binding fragments, polynucleotides, vectors, host cells, and compositions to diagnose, prevent or treat a SARS-CoV-2 infection.

### CROSS-REFERENCE TO RELATED APPLICATIONS

This application claims priority to U.S. Provisional Application Ser. No. 63/356,682, filed Jun. 29, 2022, the entire contents of which are incorporated herein by reference.

### STATEMENT OF FEDERALLY FUNDED RESEARCH

This invention was made with government support under U19AI062629-1752 awarded by the National Institutes of Health. The government has certain rights in the invention.

### TECHNICAL FIELD OF THE INVENTION

The present invention relates in general to the field of full length, fully human monoclonal antibodies, and more particularly, to human monoclonal antibodies to SARS-CoV-2.

### INCORPORATION-BY-REFERENCE OF MATERIALS FILED ON COMPACT DISC

The present application includes a Sequence Listing that has been submitted in ASCII format via EFS-Web and is hereby incorporated by reference in its entirety. Said XML copy, created on Jun. 28, 2023, is named OMRF1039.xml and is 8,487,350 bytes in size.

### BACKGROUND OF THE INVENTION

Without limiting the scope of the invention, its background is described in connection with broadly specific, neutralizing, full-length, fully human monoclonal antibodies against severe acute respiratory syndrome coronavirus 2 (SARS-CoV-2). SARS-CoV-2 is a novel Betacoronavirus that emerged in Wuhan, China, in late 2019. The rapid spread of infection globally led to the WHO declaring it a pandemic by March 2020. This novel coronavirus was initially referred to as 2019-nCoV and later renamed SARS-CoV-2 and disease caused by this virus was termed Coronavirus Disease-2019 (COVID-19) by WHO.

As of Mar. 8 2022, infection with SARS-CoV-2 has resulted in approximately 425 million COVID-19 cases and approximately 6 million deaths worldwide. The global research effort to combat this pandemic has led to rapid advances in preventative and therapeutic modalities. However, the increased incidence of breakthrough infections due to the emergence of SARS-CoV-2 variants, with mutations in their spike (S) glycoprotein, have rendered some existing therapeutics ineffective. In late 2020, a SARS-CoV-2 variant classified as Delta (B.1.617.2 and AY lineages) was identified in India, followed by the Omicron variant (B.1.1.529 and BA lineages) identified in 2021 in South Africa. Both, the Delta and Omicron variants were classified as Variants of Concern (VOC) by the Centers for Disease Control and Prevention due to their increased transmissibility, ability to cause more severe disease, significant reduction in their neutralization by Abs generated during previous infection or vaccination, reduced effectiveness of treatments or vaccines, or diagnostic detection failures (https://www.cdc.gov/coronavirus/2019-ncov/variants/variant-classifications.html). This rise in prevalence of SARS-CoV-2 variants underscores the need for effective diagnostic tools for early detection of SARS-CoV-2 infection and for interventions that can prevent and treat SARS-CoV-2 infections.

### SUMMARY OF THE INVENTION

In one embodiment, this invention includes an isolated antibody (Ab), or antigen (Ag)-binding fragment thereof, capable of binding to a SARS-CoV-2 spike (S) glycoprotein comprising: a heavy chain variable domain (VH) that comprises complementarity determining region (CDR)H1, CDRH2, and CDRH3 amino acid sequences set forth in SEQ ID NO.:1 or 11, SEQ ID NO.:2 or 12 and SEQ ID NO.:3 or 13, respectively, and a light chain variable domain (VL) that comprises CDRL1, CDRL2, and CDRL3 amino acid sequences set forth in SEQ ID NO.: 6 or 16, SEQ ID NO.: 7 or 17, and SEQ ID NO.: 8 or 18, respectively. In another aspect, the isolated Ab or Ag-binding fragment is a human Ab or a human Ag-binding fragment. In another aspect, the Ab or fragment comprises a VH sequence comprising the amino acid sequence of SEQ ID NO.: 4 or 14 and a VL sequence comprising the amino acid sequence of SEQ ID NO.: 9 or 19.

In one aspect, the SARS-CoV-2 spike (S) glycoprotein is expressed on a cell surface of a host cell; on a SARS-CoV-2 virion; or both. In another aspect, the Ab or fragment thereof binds to the spike (S) glycoprotein of: (i) a SARS-CoV-2 Wuhan-Hu-1 (GenBank QHD43416.1); (ii) a SARS-CoV-2 B.1.1.7; (iii) a SARS-CoV-2 B.1.351; (iv) a SARS-CoV-2 B.1.1.529; (v) a SARS-CoV-2 comprising any one or more of the following substitution mutations relative to amino acid sequence provided in GenBank QHD43416.1: N501Y; S477N; N439K; L452R; E484K; Y453F; A520S; K417N; K417V; S494P; N501T; 5477R; V367F; P384L; A522S; A522V; V382L; P330S; T478I; S477I; P479S; or (vi) any combination of (i)-(v). In another aspect, the Ab or fragment thereof neutralizes a SARS-CoV-2 infection: (i) in an in vitro model of infection; (ii) in an in vivo animal model of infection; (iii) in a human; or (iv) any combination of (i)-(iii).

In another aspect, the Ab or fragment thereof comprises a monoclonal Ab, a single chain Ab, a Fab, a Fab′, a F(ab′)2, a Fv, a scFv, or a scFab. In another aspect, the Ab or fragment thereof is a bivalent or bispecific Ab and comprises (a) a first target binding site that specifically binds to an epitope within the SARS-CoV-2 spike (S) polypeptide, and (b) a second target binding site that binds to a different epitope on the SARS-CoV-2 spike (S) polypeptide or a different molecule. In yet another aspect, the Ab or fragment thereof is a bivalent or bispecific Ab and comprises (a) a first target binding site that specifically binds the spike (S) glycoprotein of one of (i) a SARS-CoV-2 Wuhan-Hu-1 (GenBank QHD43416.1); (ii) a SARS-CoV-2 B.1.1.7; (iii) a SARS-CoV-2 B.1.351; or (iv) a SARS-CoV-2 comprising any one or more of the following substitution mutations relative to amino acid sequence provided in GenBank QHD43416: 1N501Y; S477N; N439K; L452R; E484K; Y453F; A520S; K417N; K417V; S494P; N501T; 5477R; V367F; P384L; A522S; A522V; V382L; P330S; T478I; S477I; P479S, and (b) a second target binding site that specifically binds the spike (S) glycoprotein of one of (i) a SARS-CoV-2 Wuhan-Hu-1 (GenBank QHD43416.1); (ii) a SARS-CoV-2 B.1.1.7; (iii) a SARS-CoV-2 B.1.351; or (iv) a SARS-CoV-2 comprising any one or more of the following substitution mutations relative to amino acid sequence provided in GenBank QHD43416: N501Y; S477N; N439K; L452R; E484K; Y453F; A520S; K417N; K417V; S494P; N501T; 5477R; V367F; P384L; A522S; A522V; V382L; P330S; T478I; S477I; P479S, not bound by (a).

In one aspect, the Ab or fragment thereof in which (i) the VH comprises or consists of an amino acid sequence having at least 85%, 90%, 95%, or 99% identity to the amino acid sequence set forth in SEQ ID NO.: 4 or 14; and/or (ii) the VL comprises or consists of an amino acid sequence having at least 85%, 90%, 95%, or 99% identity to the amino acid sequence set forth in SEQ ID NO.: 9 or 19. In another aspect, the Ab or fragment thereof is a IgG1, IgG2, IgG3, IgG4, IgA1, IgA2, IgM, IgE, or IgD isotype. In yet another aspect, the Ab or fragment thereof, further comprising (a) heavy chain constant regions (CH)1-CH3 that comprises or consists of the amino acid sequence set forth in SEQ ID NO.: 5 or 15 and (b) a light chain constant region (CL) that comprises or consists of the amino acid sequence set forth in SEQ ID NO.:10 or 20.

In one aspect, the Ab or fragment thereof binds to a receptor-binding domain (RBD) in the “up” conformation, wherein the Ab or fragment thereof is able to bind RBD in the “up” conformation and is not able to bind RBD in the “down” conformation. In another aspect, the Ab or fragment thereof binds to a RBD in the “down” conformation, wherein the Ab or fragment thereof is able to bind RBD in the “down” conformation and is not able to bind RBD in the “up” conformation.

In one aspect, the present invention comprises a hybridoma or engineered cell comprising a polynucleotide encoding the Ab or fragment thereof described above or herein.

In one aspect, the present disclosure comprises a nucleic acid molecule encoding the Ab or fragment thereof discussed above or herein. In another aspect, the present disclosure includes a vector comprising the nucleic acid molecule encoding the Ab or fragment thereof discussed above or herein.

In yet another aspect the present disclosure includes a host cell comprising the vector comprising the nucleic acid molecule encoding the Ab or fragment thereof discussed above or herein. In yet another aspect, the present invention describes a method of preparing an Ab, or Ag-binding fragment thereof, comprising: obtaining the host cell described above or herein; culturing the host cell in a medium under conditions permitting expression of the Ab or fragment thereof encoded by the vector described above or herein; and purifying the Ab or fragment thereof from the cultured cell or a medium thereof.

In one aspect, the present disclosure comprises a pharmaceutical composition comprising one or more of the Ab or fragment thereof discussed above or herein and a pharmaceutically acceptable carrier or excipient. In another aspect, the pharmaceutical composition discussed herein further comprises a second therapeutic agent. In yet another aspect, the second therapeutic agent is selected from the group consisting of: an anti-inflammatory agent, or an anti-viral agent.

In one embodiment, this invention includes a method of treating a subject infected with SARS-Cov-2 or preventing SARS-CoV-2 infection in a subject at risk of infection with SARS-CoV-2 comprising administering to a subject in need thereof a therapeutically effective amount of a SARS-CoV-2 neutralizing Ab or Ag-binding fragment. In one aspect, the Ab or Ag-binding fragment capable of binding to a SARS-CoV-2 spike (S) glycoprotein comprising: a VH sequence comprising the amino acid sequence of SEQ ID NO.: 4 or 14 or CDRH1, CDRH2, and CDRH3 amino acid sequences set forth in SEQ ID NO.:1 or 11, SEQ ID NO.:2 or 12 and SEQ ID NO.:3 or 13, respectively; and a VL sequence comprising the amino acid sequence of SEQ ID NO.: 9 or 19 or CDRL1, CDRL2, and CDRL3 amino acid sequences set forth in SEQ ID NO.: 6 or 16, SEQ ID NO.: 7 or 17, and SEQ ID NO.: 8 or 18, respectively. In one aspect, the infected subject is a mammal. In another aspect, the infected subject is human.

In one embodiment, the present disclosure comprises a method of detecting SARS-CoV-2 infection in a subject comprising the steps of: (a) contacting a sample from the subject suspected to be infected with SARS-CoV-2 with an Ab or Ag-binding fragment capable of binding to a SARS-CoV-2 spike (S) glycoprotein; and (b) detecting binding of the Ab or Ab fragment to a SARS-CoV-2 Ag in the sample. In one aspect, the Ab or Ag-binding fragment capable of binding to a SARS-CoV-2 spike (S) glycoprotein comprising: a VH sequence comprising the amino acid sequence of SEQ ID NO.: 4 or 14 or CDRH1, CDRH2, and CDRH3 amino acid sequences set forth in SEQ ID NO.:1 or 11, SEQ ID NO.:2 or 12 and SEQ ID NO.:3 or 13, respectively; and a VL sequence comprising the amino acid sequence of SEQ ID NO.: 9 or 19 or CDRL1, CDRL2, and CDRL3 amino acid sequences set forth in SEQ ID NO.: 6 or 16, SEQ ID NO.: 7 or 17, and SEQ ID NO.: 8 or 18, respectively. In one aspect, the infected subject is a mammal. In another aspect, the infected subject is human.

In one aspect, the sample in which SARS-CoV-2 infection is detected according to the present disclosure is selected from, a nasopharyngeal swab, a nares swab, saliva, urine, tears, cerebrospinal fluid, amniotic fluid, serum, plasma, whole blood, bronchopulmonary lavage, vaginal sampling and a rectal/stool sampling obtained from the subject. In yet another aspect, the SARS-CoV-2 Ag detected in the samples according to the present disclosure comprises a spike (S) glycoprotein of a human or an animal SARS-CoV-2.

In one aspect the Ab or Ab fragment contacting the sample for detection of SARS-CoV-2 infection according to the present disclosure is conjugated to at least one of: a nanoparticle, a liposome, or a detectable label. In yet another aspect, the detectable label conjugated to the Ab or Ab fragment for detection of SARS-CoV-2 infection according to the present disclosure comprises a radioactive tag, a fluorescent tag, a biological, or an enzymatic tag.

In one embodiment, the present disclosure comprises a kit for the detection of SARS-Cov-2 infection in a subject comprising: (a) an Ab or Ag-binding fragment capable of binding to a SARS-CoV-2 spike (S) glycoprotein, (b) a suitable container, and (c) an immunodetection reagent. In one aspect, the Ab or Ag-binding fragment capable of binding to a SARS-CoV-2 spike (S) glycoprotein comprising: a VH sequence comprising the amino acid sequence of SEQ ID NO.: 4 or 14 or CDRH1, CDRH2, and CDRH3 amino acid sequences set forth in SEQ ID NO.:1 or 11, SEQ ID NO.:2 or 12 and SEQ ID NO.:3 or 13, respectively; and a VL sequence comprising the amino acid sequence of SEQ ID NO.: 9 or 19 or CDRL1, CDRL2, and CDRL3 amino acid sequences set forth in SEQ ID NO.: 6 or 16, SEQ ID NO.: 7 or 17, and SEQ ID NO.: 8 or 18, respectively. In one aspect, the subject is a mammal. In another aspect, the subject is human.

In another aspect, the Ab or fragment thereof, comprised in the kit according to the present disclosure and as discussed above or herein, are affixed to a support selected from one or more beads, a dipstick, a filter, a membrane, a plate, a chip, or a column matrix. In yet another aspect, the immunodetection reagent, comprised in the kit according to the present disclosure, comprises at least a second Ab that binds an immunocomplex formed when the Ab or Ab fragment discussed above binds SARS-CoV-2 or SARS-CoV-2 Ag. In yet another aspect, the kit for the detection of SARS-CoV-2 infection in a subject, according to the present disclosure further comprises SARS-CoV-2 or SARS-CoV-2 Ag for use as a standard.

In one aspect, at least one of the Ab or Ab fragment, or the immunodetection reagent, comprised in the kit according to the present disclosure, are linked to a detectable label. In another aspect, the detectable label linked to the Ab or Ab fragment, or the immunodetection reagent, according to the present disclosure, comprises a radioactive tag, a fluorescent tag, a biological, or an enzymatic tag.

### BRIEF DESCRIPTION OF THE DRAWINGS

For a more complete understanding of the features and advantages of the present invention, reference is now made to the detailed description of the invention along with the accompanying figures and in which:

FIG. 1 shows a pseudovirus/lentiviral neutralization assay conducted using commercial S-pseudotyped lentivirus in a 96-well format.

FIG. 2 shows SARS-CoV-2 neutralization assay where antibody was serially diluted 1:2 across a 96-well plate and mixed with enough virus (isolate USA-WA1/2020).

FIG. 3 shows the amino acid sequence for the framework regions (FR) 1-3, CDR 1-3 and joining (J) regions of the heavy and light chains of antibodies designated 810p2C01 and 810p3CO3 that have SARS-CoV-2 binding capacity. The figure also provides the genes utilized for the variable (V), diversity (D) and joining (J) regions of the heavy chains and the genes utilized in the V and J region of the light chains of the two antibodies. SEQ ID NOS:4, 9, 14, and 19, are as indicated in the figure, with the framework regions and CDRs labeled, as shown.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

The present invention relates to novel mAbs that bind to the SARS-CoV-2 spike (S) glycoprotein. The present invention provides such mAbs and Ab fragments thereof, which are useful for detection or prevention and/or treatment of SARS-CoV-2. The present invention also provides a pharmaceutical composition comprising the novel MAbs or Ab fragments thereof. In addition, the present invention provides a kit and method for detecting SARS-CoV-2 and a method for preventing or treating SARS-CoV-2, using the novel MAbs or Ab fragments thereof as described herein.

SARS-CoV-2 belongs to the Betacoronavirus genus, a group of related RNA viruses that can cause fatal respiratory tract infections. This genus includes the severe acute respiratory syndrome coronavirus (SARS-CoV), Middle East respiratory syndrome (MERS), and SARS-CoV-2 [1]. Only four common mildly pathogenic coronaviruses are endemic to humans, including HCoV-0C43, HCoV-HKU1, HCoV-NL63, and HCoV-229E [2]. Both MERS and SARS-CoV are zoonotic pathogens transmitted to humans from intermediate hosts such as dromedary camels and civet cats, respectively [3,4]. While the origin of SARS-CoV-2 is still unclear, bats are the likely to be the reservoir hosts with other mammalian species acting as intermediaries to promote the zoonotic transfer [5].

These coronaviruses are all enveloped viruses with a positive-sense single-stranded RNA genome surrounded by nucleocapsid phosphoproteins. The coronavirus virion is made up of the nucleocapsid (N), membrane (M), envelope (E) and spike (S) proteins, which are structural proteins. The coronavirus S protein consists of two subunits, S1 and S2. S1 subunit contains a receptor-binding domain (RBD) which mediates binding to the receptor on host cell, whereas S2 plays a role in membrane fusion. Angiotensin-converting enzyme 2 (ACE2) was previously shown to be a crucial receptor for SARS-CoV infection in vivo [6], and was rapidly identified as a receptor for SARS-CoV-2. Although several SARS-CoV-2 candidate receptors have been identified in addition to ACE2, whether these receptor interactions are biologically relevant remains to be established. Upon binding of SARS-CoV-2 particles to receptors on target cells, the S protein is cleaved by host proteases leading to fusion of viral and cellular membranes. In the absence of cellular proteases, coronaviruses enter host cells through the endocytic pathways where fusion is induced by low pH and S cleavage by endosomal/lysosomal proteases (cathepsins) [reviewed in refs. 7,8]. Following fusion, the nucleocapsid is released into the cytoplasm and the genomic RNA is translated after dissociation from N. Viral genomes, coated with N protein bud into the lumen of the ER-Golgi intermediate compartment to form enveloped particles containing M, E and S proteins and the progeny virions are finally trafficked to the cell surface for release.

SARS-CoV-2 results in a disease called COVID-19, which involves both pneumonia and acute respiratory distress syndrome (ARDS). COVID-19 can also lead to complications such as acute liver, cardiac and kidney injury, as well as secondary infection and inflammatory response. Upon infection with an RNA virus, the process of innate immune sensing begins with the engagement of pattern recognition receptors (PRRs) by viral RNA. Activation of PRR leads to secretion of anti-viral cytokines such as type I/III interferons (IFNs) and other cytokines such as proinflammatory tumor necrosis factor alpha (TNF-α), and interleukin-1 (IL-1), IL-6, and IL-18. These cytokines can then potentiate an adaptive immune response including activation of T cells and B cells. A cytotoxic CD8+ T cell response typically observed within 7 days of symptoms and peaking at 14 days, correlates with mild disease and while type 1 CD4+ phenotype is associated with an effective viral control, a type 2 profile is often seen in those with severe disease [9]. Activation of B cells lead to antibody (Ab) responses predominantly against S (particularly RBD) and N proteins. The median time to seroconversion for anti-RBD Abs is 11-13 days after the onset of symptoms with decline in anti-RBD IgG observed around 5-8 months [10]. However, the existence and duration of a memory response to SARS-CoV-2 is yet to be clearly established.

In severe COVID-19, the immune system over activated and causes a ‘cytokine storm’ characterized by the release of high levels of cytokines, especially IL-6 and TNF-α, into the circulation, causing a local and systemic inflammatory response [11, 12]. Additionally, there is release of pro-IL-1β, which is cleaved into the active mature IL-1β that mediates lung inflammation and ultimately, fibrosis [13]. Although the respiratory system is the principal target for SARS-CoV-2, it can affect the gastrointestinal tract (GI), hepatobiliary, cardiovascular, renal, and central nervous system. Either one or a combination of mechanisms explains SARS-CoV-2—induced organ dysfunction: direct viral toxicity, ischemic injury caused by vasculitis, thrombosis, or thrombo-inflammation, immune dysregulation, and renin-angiotensin-aldosterone system (RAAS) dysregulation [14]. The median incubation period for SARS-CoV-2 is around 5.1 days, and the majority of patients develop symptoms within 11.5 days of infection [15]. The clinical spectrum of COVID-19 varies from asymptomatic or mildly symptomatic forms to clinical illness characterized by acute respiratory failure requiring mechanical ventilation, septic shock, and multiple organ failure.

Diagnosis: The standard test for diagnosing a SARS-CoV-2 infection is testing a nasopharyngeal swab for SARS-CoV-2 nucleic acid using a real-time PCR assay. The US Food and Drug Administration (FDA) has provided emergency use authorizations (EUAs) for commercial PCR assays validated for the qualitative detection of SARS-CoV-2 nucleic acid in other specimen such as, oropharyngeal swabs, anterior/mid-turbinate nasal swabs, nasopharyngeal aspirates, bronchoalveolar lavage (BAL) and saliva. An antibody test can evaluate for the presence of antibodies that are generated as a result of infection. Antibody tests play an important role in broad-based surveillance of COVID-19, and many commercial manufactured antibody testing kits are available to evaluate the presence of antibodies against SARS-CoV-2 are available. Considering this viral illness commonly manifests itself as pneumonia, radiological imaging has a fundamental role in the diagnostic process, management, and follow-up. Imaging studies may include chest x-ray, lung ultrasound, or chest computed tomography (CT).

Treatment: Several therapeutic options to treat COVID-19 are currently available. These include antiviral drugs (e.g., molnupiravir, paxlovid, remdesivir), anti-SARS-CoV-2 mAbs (e.g., bamlanivimab/etesevimab, casirivimab/imdevimab), anti-inflammatory drugs (e.g., dexamethasone), and immunomodulator agents (e.g., baricitinib, tocilizumab) that are available under FDA issued EUA or are being evaluated in the management of COVID-19 [14]. Antiviral medications and Ab-based treatments are likely to be more effective during the early phase of infection when viral replication is at its peak while anti-inflammatory drugs, immunomodulating therapies, or a combination of these therapies may help combat the hyperinflammatory, prothrombotic state observed in the later phase.

The invention described herein is based, at least in part, on the disclosed anti-SARS-CoV-2 Abs or Ab fragments thereof and their unexpected activity to broadly bind and neutralize SARS-CoV-2. These Abs and Ab fragments constitute a novel strategy in treating and preventing SARS-CoV-2 infections.

As used herein, the term “amino acid” refers to naturally occurring and synthetic amino acids, as well as amino acid analogs and amino acid mimetics that function in a manner similar to the naturally occurring amino acids. Naturally, occurring amino acids are those encoded by the genetic code, as well as those amino acids that are later modified, e.g., hydroxyproline, γ-carboxyglutamate, and O-phosphoserine. Amino acid analogs refer to compounds that have the same basic chemical structure as a naturally occurring amino acid, i.e., an α-carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refer to chemical compounds that have a structure that is different from the general chemical structure of an amino acid, but that functions in a manner similar to a naturally occurring amino acid.

As used herein, “antibody” or “Antigen-binding fragment” refers to immunoglobulin molecules and immunologically active portions of immunoglobulin molecules, i.e., molecules that contain an antigen (Ag) binding portion that immunospecifically binds a glycoprotein. As such, the term antibody (Ab) encompasses not only whole Ab molecules, but also Ab fragments as well as variants (including derivatives) of Abs and Ab fragments. In natural Abs, two heavy chains are linked to each other by disulfide bonds and each heavy chain is linked to a light chain by a disulfide bond. There are two types of light chain, lambda (1) and kappa (κ). Five main heavy chain classes (or isotypes) determine the functional activity of an Ab molecule: IgM, IgD, IgG, IgA and IgE. Each chain contains distinct sequence domains. Modified versions of each of these classes and isotypes are readily discernable to the skilled artisan in view of the instant disclosure and, accordingly, are within the scope of the instant disclosure. All immunoglobulin classes are clearly within the scope of the present disclosure, the following discussion will generally be directed to the IgG class of immunoglobulin molecules. With regard to IgG, a standard immunoglobulin molecule comprises two identical light chain polypeptides of molecular weight approximately 23,000 Daltons, and two identical heavy chain polypeptides of molecular weight 53,000-70,000.

The light chain includes two domains, a variable domain (VL) and a constant domain (CL). The heavy chain includes four domains, a variable domain (VH) and three constant domains (CH1, CH2 and CH3, collectively referred to as CH). The variable regions of both light (VL) and heavy (VH) chains determine binding recognition and specificity to the Ag. The light and heavy chains of an Ab each have three complementarity determining regions (CDRs), designated LCDR1, LCDR2, LCDR3 and HCDR1, HCDR2, HCDR3, respectively. An Ag-binding site, therefore, includes six CDRs, comprising the CDR set from each of a heavy and a light chain variable region. Framework Regions (FRs) refer to amino acid sequences interposed between CDRs.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g., monovalent nanobodies, bivalent nanobodies, etc.), small modular immunopharmaceuticals (SMIPs), and shark variable IgNAR domains, are also encompassed within the expression “antigen-binding fragment,” as used herein.

An antigen-binding fragment of an antibody will typically comprise at least one variable domain. The variable domain may be of any size or amino acid composition and will generally comprise at least one CDR which is adjacent to or in frame with one or more framework sequences. In antigen-binding fragments having a V H domain associated with a VL domain, the VH and VL domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V H or V L domain. In certain embodiments, an Ab or Ag-binding fragment of the present disclosure specifically binds to a SARS-CoV-2 Ag or to a SARS-CoV-2 spike (S) glycoprotein or polypeptide.

As used herein, the term “Antigen” (Ag), as used herein, refers to an immunogenic molecule that provokes an immune response. This immune response may involve Ab production, activation of specific immunologically-competent cells, activation of complement, Ab-dependent cytotoxicicity, or any combination thereof. An antigen (Ag-immunogenic molecule) may be, for example, a peptide, glycopeptide, polypeptide, glycopolypeptide, polynucleotide, polysaccharide, lipid, or the like. It is readily apparent that an Ag can be synthesized, produced recombinantly, or derived from a biological sample. Exemplary biological samples that can contain one or more Ags include tissue samples, stool samples, cells, biological fluids, or combinations thereof. Ags can be produced by cells that have been modified or genetically engineered to express an Ag. Ags can also be present in a SARS-CoV-2 (e.g., a surface or a spike glycoprotein or portion thereof), such as present in a virion, or expressed or presented on the surface of a cell infected by SARS-CoV-2.

As used herein, “anti-inflammatory agent” includes any therapeutic drug or substance that reduces inflammation (redness, swelling, and pain) in the body. The one or more anti-inflammatory agents for use in a combination therapy of the present disclosure comprises a corticosteroid such as, for example, dexamethasone, prednisone, or the like. In some embodiments, the one or more anti-inflammatory agents comprise a cytokine antagonist such as, for example, an antibody that binds to IL6 (such as siltuximab), or to IL-6R (such as tocilizumab), or to IL-1β, IL-7, IL-8, IL-9, IL-10, FGF, G-CSF, GM-CSF, IFN-γ, IP-10, MCP-1, MIP-1A, MIP1-B, PDGR, TNF-α, or VEGF. In some embodiments, anti-inflammatory agents such as ruxolitinib and/or anakinra are used. Anti-inflammatory agents for use in a combination therapy of the present disclosure also include non-steroidal anti-inflammatory drugs (NSAIDS).

As used herein, “anti-viral agent” includes any drug or substance to treat a viral infection. The one or more anti-viral agents for use in a combination therapy of the present disclosure comprises nucleotide analogs or nucelotide analog prodrugs such as, for example, remdesivir, sofosbuvir, acyclovir, and zidovudine. The anti-viral agent also includes lopinavir, ritonavir, favipiravir, or any combination thereof. In certain embodiments, the anti-viral agent administered with the Ab or Ag-binding fragment of the present disclosure comprises leronlimab.

As used herein, the phrase “bispecific antibody” or “bivalent antibody” includes an antibody (Ab) capable of selectively binding two or more epitopes. Bispecific Abs include fragments of two different monoclonal Abs and generally comprise two nonidentical heavy chains derived from the two different monoclonal Abs, with each heavy chain specifically binding a different epitope—either on two different molecules (e.g., different epitopes on two different immunogens) or on the same molecule. Bispecific Abs can be made, for example, by combining heavy chains that recognize different epitopes of the same or different immunogen. For example, nucleic acid sequences encoding heavy chain variable sequences that recognize different epitopes of the same or different immunogen can be fused to nucleic acid sequences encoding the same or different heavy chain constant regions, and such sequences can be expressed in a cell that expresses an immunoglobulin light chain. A typical bispecific Ab has two heavy chains each having three heavy chain CDRs, followed by (N-terminal to C-terminal) a CH1 domain, a hinge, a CH2 domain, and a CH3 domain, and an immunoglobulin light chain that either does not confer epitope-binding specificity but that can associate with each heavy chain, or that can associate with each heavy chain and that can bind one or more of the epitopes bound by the heavy chain epitope-binding regions, or that can associate with each heavy chain and enable binding or one or both of the heavy chains to one or both epitopes.

As used herein, “epitope” or “antigenic epitope” includes any molecule, structure, amino acid sequence, or protein determinant that is recognized and specifically bound by a cognate binding molecule, such as an immunoglobulin, or other binding molecule, domain, or protein. Epitopic determinants generally contain chemically active surface groupings of molecules, such as amino acids or sugar side chains, and can have specific three-dimensional structural characteristics, as well as specific charge characteristics. Where an Ag comprises a peptide or protein, the epitope can be comprised of consecutive amino acids (e.g., a linear epitope), or can be comprised of amino acids from different parts or regions of the protein that are brought into proximity by protein folding (e.g., a discontinuous or conformational epitope), or non-contiguous amino acids that are in close proximity irrespective of protein folding.

As used herein, the term “host” refers to a cell or microorganism targeted for genetic modification with a heterologous nucleic acid molecule to produce a polypeptide of interest (e.g., an antibody of the present disclosure). A host cell may include any individual cell or cell culture which may receive a vector or the incorporation of nucleic acids or express proteins. The term also encompasses progeny of the host cell, whether genetically or phenotypically the same or different. Suitable host cells may depend on the vector and may include mammalian cells, animal cells, human cells, simian cells, insect cells, yeast cells, and bacterial cells. These cells may be induced to incorporate the vector or other material by use of a viral vector, transformation via calcium phosphate precipitation, DEAE-dextran, electroporation, microinjection, or other methods. See, for example, Sambrook et al., Molecular Cloning: A Laboratory Manual 2d ed. (Cold Spring Harbor Laboratory, 1989).

As used herein, the term “Fab” (fragment antigen binding) refers to the part of an Ab that binds to Ag and includes the variable region and CH1 of the heavy chain linked to the light chain via an inter-chain disulfide bond. Each Fab fragment is monovalent with respect to Ag binding, i.e., it has a single Ag-binding site. Pepsin treatment of an Ab yields a single large F(ab′)2 fragment that roughly corresponds to two disulfide linked Fab fragments having divalent Ag-binding activity and is still capable of cross-linking Ag. Both the Fab and F(ab′)2 are examples of “Ag-binding fragments.” Fab′ fragments differ from Fab fragments by having additional few residues at the carboxy terminus of the CH1 domain including one or more cysteines from the Ab hinge region. Fab′-SH is the designation herein for Fab′ in which the cysteine residue(s) of the constant domains bear a free thiol group. F(ab′)2 Ab fragments originally were produced as pairs of Fab′ fragments that have hinge cysteines between them. Other chemical couplings of Ab fragments are also known. Fab fragments may be joined, e.g., by a peptide linker, to form a single chain Fab, also referred to herein as “scFab.” In these embodiments, an inter-chain disulfide bond that is present in a native Fab may not be present, and the linker serves in full or in part to link or connect the Fab fragments in a single polypeptide chain.

As used herein, the term “Fv” is a small Ab fragment that contains a complete Ag-recognition and Ag-binding site. This fragment generally consists of a dimer of one heavy- and one light-chain variable region domain in tight, non-covalent association. However, even a single variable domain (or half of an Fv comprising only three CDRs specific for an Ag) has the ability to recognize and bind Ag, although typically at a lower affinity than the entire binding site.

As used herein, the term “Single-chain Fv” also abbreviated as “sFv” or “scFv”, are Ab fragments that comprise the VH and VL Ab domains connected into a single polypeptide chain. In some embodiments, the scFv polypeptide comprises a polypeptide linker disposed between and linking the VH and VL domains that enables the scFv to retain or form the desired structure for Ag binding. Such a peptide linker can be incorporated into a fusion polypeptide using standard techniques well known in the art. Additionally or alternatively, Fv can have a disulfide bond formed between and stabilizing the VH and the VL. For a review of scFv, see Pluckthun in The Pharmacology of Monoclonal Antibodies, vol. 113, Rosenburg and Moore eds., Springer-Verlag, New York, pp. 269-315 (1994); Borrebaeck 1995, infra. In certain embodiments, the Ab or Ag-binding fragment comprises a scFv comprising a VH domain, a VL domain, and a peptide linker linking the VH domain to the VL domain. In particular embodiments, a scFv comprises a VH domain linked to a VL domain by a peptide linker, which can be in a VH-linker-VL orientation or in a VL-linker-VH orientation. Any scFv of the present disclosure may be engineered so that the C-terminal end of the VL domain is linked by a short peptide sequence to the N-terminal end of the VH domain, or vice versa (i.e., (N)VL(C)-linker-(N)VH(C) or (N)VH(C)-linker-(N)VL(C). Alternatively, in some embodiments, a linker may be linked to an N-terminal portion or end of the VH domain, the VL domain, or both. scFv can be constructed using any combination of the VH and VL sequences or any combination of the CDRH1, CDRH2, CDRH3, CDRL1, CDRL2, and CDRL3 sequences disclosed herein.

As used herein, a “monoclonal antibody” refers to an Ab obtained from a population of substantially homogeneous Abs, e.g., the individual Abs comprising the population are identical except for possible naturally-occurring mutations that may be present in minor amounts. In contrast to polyclonal Ab preparations, which typically include different Abs directed against different determinants (epitopes), each monoclonal Ab is directed against a single determinant on the Ag (epitope). The modifier “monoclonal” indicates the character of the Ab as being obtained from a substantially homogeneous population of Abs, and is not to be construed as requiring production of the Ab by any particular method. For example, the monoclonal Abs to be used in accordance with the present invention may be made by the hybridoma method first described by Kohler and Milstein, 1975, Nature, 256:495, or may be made by recombinant DNA methods such as described in U.S. Pat. No. 4,816,567. The monoclonal Abs may also be isolated from phage libraries generated using the techniques described in McCafferty et al., 1990, Nature, 348:552-554, for example. Other methods are known in the art and are contemplated for use herein.

As used herein, “neutralizing antibody” is an Ab that can neutralize, i.e., prevent, inhibit, reduce, impede, or interfere with, the ability of a pathogen to initiate and/or perpetuate an infection in a host. The terms “neutralizing Ab” and “an Ab that neutralizes” or “Abs that neutralize” are used interchangeably herein. In any of the presently disclosed embodiments, the Ab or Ag-binding fragment is capable of preventing and/or neutralizing a SARS-CoV-2 infection in an in vitro model of infection and/or in an in vivo animal model of infection and/or in a human.

As used herein, the term “Nucleic acid molecule” or “polynucleotide” or “polynucleic acid” refers to a polymeric compound including covalently linked nucleotides, which can be made up of natural subunits (e.g., purine or pyrimidine bases) or non-natural subunits (e.g., morpholine ring). Purine bases include adenine, guanine, hypoxanthine, and xanthine, and pyrimidine bases include uracil, thymine, and cytosine. Nucleic acid molecules include polyribonucleic acid (RNA), which includes mRNA, microRNA, siRNA, viral genomic RNA, and synthetic RNA, and polydeoxyribonucleic acid (DNA), which includes cDNA, genomic DNA, and synthetic DNA, either of which may be single or double stranded. If single-stranded, the nucleic acid molecule may be the coding strand or non-coding (anti-sense) strand. A nucleic acid molecule encoding an amino acid sequence includes all nucleotide sequences that encode the same amino acid sequence. Some versions of the nucleotide sequences may also include intron(s) to the extent that the intron(s) would be removed through co- or post-transcriptional mechanisms. In other words, different nucleotide sequences may encode the same amino acid sequence as the result of the redundancy or degeneracy of the genetic code, or by splicing.

Variants of nucleic acid molecules of this disclosure are also contemplated. Variant nucleic acid molecules are at least 70%, 75%, 80%, 85%, 90%, and are preferably 95%, 96%, 97%, 98%, 99%, or 99.9% identical a nucleic acid molecule of a defined or reference polynucleotide as described herein, or that hybridize to a polynucleotide under stringent hybridization conditions of 0.015M sodium chloride, 0.0015M sodium citrate at about 65-68° C. or 0.015M sodium chloride, 0.0015M sodium citrate, and 50% formamide at about 42° C. Nucleic acid molecule variants retain the capacity to encode a binding domain thereof having a functionality described herein, such as binding a target molecule.

As used herein, the term “percent sequence identity” refers to a relationship between two or more sequences, as determined by comparing the sequences. Preferred methods to determine sequence identity are designed to give the best match between the sequences being compared. For example, the sequences are aligned for optimal comparison purposes (e.g., gaps can be introduced in one or both of a first and a second amino acid or nucleic acid sequence for optimal alignment). Further, non-homologous sequences may be disregarded for comparison purposes. The percent sequence identity referenced herein is calculated over the length of the reference sequence, unless indicated otherwise. Methods to determine sequence identity and similarity can be found in publicly available computer programs. Sequence alignments and percent identity calculations may be performed using a BLAST program (e.g., BLAST 2.0, BLASTP, BLASTN, or BLASTX). The mathematical algorithm used in the BLAST programs can be found in Altschul et al., Nucleic Acids Res. 25:3389-3402, 1997. Within the context of this disclosure, it will be understood that where sequence analysis software is used for analysis, the results of the analysis are based on the “default values” of the program referenced. “Default values” mean any set of values or parameters which originally load with the software when first initialized.

As used herein, the term “pharmaceutical composition” comprises the combination of an active agent, such as any one or more of the presently disclosed Abs, Ag-binding fragments, polynucleotides, peptides, vectors, or host cells, singly or in any combination, and can further comprise a pharmaceutically acceptable carrier, excipient, or diluent, inert or active, in a sterile composition suitable for diagnostic or therapeutic use in vitro, in vivo or ex vivo. Carriers, excipients, and diluents are discussed in further detail herein. Such compositions comprising the disclosed Ab, Ag-binding fragment, polynucleotide, vector, host cell, peptides, or composition of the present disclosure may also be administered simultaneously with, prior to, or after administration of one or more other therapeutic agents such as anti-viral or anti-inflammatory agents.

As used herein, the term “pharmaceutically acceptable carrier” or “pharmaceutical acceptable excipient” includes any material which, when combined with an active ingredient, allows the ingredient to retain biological activity and is non-reactive with the subject's immune system. This further includes materials from such compounds that are appropriate for use in pharmaceutical contexts, i.e., materials which are, within the scope of sound medical judgment, suitable for use in contact with the tissues of humans and lower animals without undue toxicity, irritation, allergic response and the like, and are commensurate with a reasonable benefit/risk ratio. Examples include, but are not limited to, any of the standard pharmaceutical carriers such as a phosphate buffered saline solution, water, emulsions such as oil/water emulsion, and various types of wetting agents. Preferred diluents for aerosol or parenteral administration are phosphate buffered saline or normal (0.9%) saline. Compositions comprising such carriers are formulated by well-known conventional methods (see, for example, Remington's Pharmaceutical Sciences, 18th edition, A. Gennaro, Ed., Mack Publishing Co., Easton, Pa., 1990; and Remington, The Science and Practice of Pharmacy 20th Ed. Mack Publishing, 2000).

As used herein, the term “protein” or “polypeptide” refers to a polymer of amino acid residues. Proteins apply to naturally occurring amino acid polymers, as well as to amino acid polymers in which one or more amino acid residue is an artificial chemical mimetic of a corresponding naturally occurring amino acid, and non-naturally occurring amino acid polymers. Variants of proteins, peptides, and polypeptides of this disclosure are also contemplated. In certain embodiments, variant proteins, peptides, and polypeptides comprise or consist of an amino acid sequence that is at least 70%, 75%, 80%, 85%, 90%, 91%, 92%, 93%, 94%, 95%, 96%, 97%, 98%, 99%, or 99.9% identical to an amino acid sequence of a defined or reference amino acid sequence as described herein.

As used herein, the term “SARS-CoV-2”, also referred to herein as “Wuhan coronavirus”, or “Wuhan seafood market pneumonia virus”, or “Wuhan CoV”, or “novel CoV”, or “nCoV”, or “2019 nCoV”, or “Wuhan nCoV” is a betacoronavirus believed to be of lineage B (sarbecovirus). SARS-CoV-2 was first identified in Wuhan, Hubei province, China, in late 2019 and spread within China and to other parts of the world by early 2020. Symptoms of SARS-CoV-2 include fever, dry cough, and dyspnea. The genomic sequence of SARS-CoV-2 isolate Wuhan-Hu-1 is provided in GenBank MN908947.3 and the amino acid translation of the genome is provided in GenBank QHD43416.1. Like other coronaviruses (e.g., SARS CoV), SARS-CoV-2 comprises a “spike” or surface (“S”) type I transmembrane glycoprotein containing a receptor binding domain (RBD). RBD is believed to mediate entry of the lineage B SARS coronavirus to respiratory epithelial cells by binding to the cell surface receptor angiotensin-converting enzyme 2 (ACE2). In particular, a receptor binding motif (RBM) in the virus RBD is believed to interact with ACE2. Ab and Ag-binding fragments of the present disclosure are capable of binding to a SARS CoV-2 surface glycoprotein (S), such as that of Wuhan-Hu-1. For example, in certain embodiments, an Ab or Ag-binding fragment binds to an epitope in Wuhan-Hu-1 S protein RBD. SARS-CoV-2 Wuhan-Hu-1 S protein has approximately 73% amino acid sequence identity with SARS-CoV S protein. SARS-CoV-2 RBD has approximately 75% to 77% amino acid sequence similarity to SARS coronavirus RBD, and SARS-CoV-2 Wuhan Hu-1 RBM has approximately 50% amino acid sequence similarity to SARS coronavirus RBM. Unless otherwise indicated herein, SARS-CoV-2 Wuhan Hu-1 refers to a virus comprising the amino acid sequence set forth in GenBank QHD43416.1, optionally with the genomic sequence set forth in GenBank MN908947.3. There have been a number of emerging SARS-CoV-2 variants. Some SARS-CoV-2 variants contain an N439K mutation, which has enhanced binding affinity to the human ACE2 receptor [16]. Some SARS-CoV-2 variants contain an N501Y mutation, which is associated with increased transmissibility, including the lineages B.1.1.7 (also known as 20I/501Y.Vi1 and VOC 202012/01; (de169-70, de1144, N501Y, A570D, D614G, P681H, T7161, S982A, and D1118H mutations)) and B.1.351 (also known as 20H/501Y.V2; L18F, D80A, D215G, R246I, K417N, E484K, N501Y, D614G, and A701V mutations), which were discovered in the United Kingdom and South Africa, respectively [17, 18]. B.1.351 also include two other mutations in the RBD domain of SARS-CoV2 spike protein, K417N and E484K [17]. Other SARS-CoV-2 variants include the Lineage B.1.1.28, which was first reported in Brazil; the Variant P.1, lineage B.1.1.28 (also known as 20J/501Y.V3), which was first reported in Japan; Variant L452R, which was first reported in California in the United States (Pan American Health Organization, Epidemiological update: Occurrence of variants of SARS-CoV-2 in the Americas, Jan. 20, 2021, available at reliefweb.int/sites/reliefweb.int/files/resources/2021-jan-20-phe-epi-update-SARS-CoV-2.pdf). Other SARS-CoV-2 variants include a SARS CoV-2 of Glade 19A; SARS CoV-2 of Glade 19B; a SARS CoV-2 of Glade 20A; a SARS CoV-2 of Glade 20B; a SARS CoV-2 of Glade 20C; a SARS CoV-2 of Glade 20D; a SARS CoV-2 of Glade 20E (EU1); a SARS CoV-2 of Glade 20F; a SARS CoV-2 of Glade 20G; and SARS CoV-2 B1.1.207; and other SARS CoV-2 lineages [19]. Another variant of SARS-CoV-2, B.1.1.529 (Omicron) was first reported to the World Health Organization (WHO) by South Africa on Nov. 24, 2021 [20]. The foregoing SARS-CoV-2 variants, and the amino acid and nucleotide sequences thereof, are incorporated herein by reference.

As used herein, the term “specifically binds” refers to an association or union of an antibody or antigen-binding fragment to an antigen with an affinity or Kₐ (i.e., an equilibrium association constant of a particular binding interaction with units of 1/M) equal to or greater than 10⁵ M⁻¹ (which equals the ratio of the on-rate [Kₒₙ] to the off rate [K_{off}] for this association reaction), while not significantly associating or uniting with any other molecules or components in a sample. Alternatively, affinity may be defined as an equilibrium dissociation constant (K d) of a particular binding interaction with units of M (e.g., 10⁻⁵ M to 10⁻¹³ M). Antibodies may be classified as “high-affinity” antibodies or as “low-affinity” antibodies. “High-affinity” antibodies refer to those antibodies having a Kₐ of at least 10⁷ M⁻¹, at least 10⁸ M⁻¹, at least 10⁹ M⁻¹, at least 10¹⁰ M⁻¹, at least 10¹¹ M⁻¹, at least 10¹² M⁻¹, or at least 10¹³ M⁻¹. “Low-affinity” antibodies refer to those antibodies having a Kₐ of up to 10⁷ M⁻¹, up to 10⁶ M⁻¹, up to 10⁵ M⁻¹. Alternatively, affinity may be defined as an equilibrium dissociation constant (K a) of a particular binding interaction with units of M (e.g., 10⁻⁵ M to 10⁻¹³ M).

In some contexts, Ab and Ag-binding fragments may be described with reference to affinity and/or to avidity for antigen. Unless otherwise indicated, avidity refers to the total binding strength of an Ab or Ag-binding fragment thereof to Ag, and reflects binding affinity, valency of the Ab or antigen-binding fragment (e.g., whether the antibody or antigen-binding fragment comprises one, two, three, four, five, six, seven, eight, nine, ten, or more binding sites), and, for example, whether another agent is present that can affect the binding (e.g., a non-competitive inhibitor of the antibody or antigen-binding fragment).

As used herein, the phrases “therapeutically effective amount” and “therapeutic level” mean that drug dosage or plasma concentration in a subject, respectively, that provides the specific pharmacological effect for which the drug is administered in a subject in need of such treatment, i.e., to reduce, ameliorate, or eliminate the symptoms or effects of SARS-CoV-2 infection. It is emphasized that a therapeutically effective amount or therapeutic amount of an antibody will not always be effective in treating the conditions/diseases described herein, even though such dosage is deemed to be a therapeutically effective amount by those of skill in the art. The therapeutically effective amount may vary based on the route of administration and dosage form, the age and weight of the subject, and/or the subject's condition, including the SARS-CoV-2 variant and severity of the infection, among other factors. The human subject treated according to the present disclosure include an infant, a child, a young adult, an adult of middle age, or an elderly person. The human subject treated according to the present disclosure include those less than 1 year old, or those 1 to 5 years old, or those between 5 and 125 years old (e.g., 5, 10, 15, 20, 25, 30, 35, 40, 45, 50, 55, 60, 65, 70, 75, 80, 90, 95, 100, 105, 110, 115, or 125 years old, including any and all ages therein or there between). The human subject treated includes male and female.

As used herein, the term “treat”, “treating”, and “treatment” refer to therapeutic or preventative measures described herein. The methods of “treatment” employ administration of an Ab to a subject having a disease or disorder, or predisposed to having such a disease or disorder, in order to prevent, cure, delay, reduce the severity of, or ameliorate, partially or completely alleviate one or more symptoms of the disease or disorder or recurring disease or disorder, or in order to prolong the survival of a subject beyond that expected in the absence of such treatment. Treatment may be administered to a subject who does not exhibit signs of a disease, disorder, and/or condition or to a subject who exhibits only early signs of the disease, disorder, and/or condition, for example, for the purpose of decreasing the risk of developing pathology associated with the disease, disorder, and/or condition.

As used herein, the term “vector” or “expression vector” refers to a DNA construct containing a nucleic acid molecule that is operably linked to a suitable control sequence capable of effecting the expression of the nucleic acid molecule in a suitable host. Such control sequences include a promoter to effect transcription, an optional operator sequence to control such transcription, a sequence encoding suitable mRNA ribosome binding sites, and sequences which control termination of transcription and translation. The vector may be a plasmid, a phage particle, a virus, or simply a potential genomic insert. Once transformed into a suitable host, the vector may replicate and function independently of the host genome, or may, in some instances, integrate into the genome itself or deliver the polynucleotide contained in the vector into the genome without the vector sequence. In the present specification, “plasmid,” “expression plasmid,” “virus,” and “vector” are often used interchangeably.

General Methods for monoclonal antibody production: It will be understood that monoclonal Abs binding to SARS-CoV-2 will have utility in several applications. These include the production of diagnostic kits for use in detecting and diagnosing disease. In these contexts, one may link such Abs to diagnostic or therapeutic agents, or use them as capture agents or competitors in competitive assays. Means for preparing and characterizing Abs are well known in the art (see, e.g., Antibodies: A Laboratory Manual, Cold Spring Harbor Laboratory, 1988; U.S. Pat. No. 4,196,265).

The methods for generating monoclonal Abs (MAbs) generally begin along the same lines as those for preparing polyclonal Abs. The first step for both these methods is immunization of an appropriate host or identification of subjects who are immune due to prior natural infection. In the case of human monoclonal Abs, one may instead simply look for an individual already known to have generated an immune response, in this case, to have been exposed to SARS-CoV-2 or immunized with vaccines to prevent SARS-COV-2, such as COMIRNATY®, Spikevax™, or others.

Monoclonal Abs useful in the present invention may be prepared by the hybridoma methodology first described by Kohler et al., Nature 256:495 (1975), or may be made using recombinant DNA methods in bacterial, eukaryotic animal, or plant cells (see, e.g., U.S. Pat. No. 4,816,567). Monoclonal Abs may also be isolated from phage antibody libraries using the techniques described in Clackson et al., Nature, 352:624-628 (1991) and Marks et al., J. Mol. Biol., 222:581-597 (1991), for example. Monoclonal Abs may also be obtained using methods disclosed in PCT Publication No. WO 2004/076677A2.

It also is contemplated that a molecular cloning approach may be used to generate monoclonal Abs. For this, RNA can be isolated from the hybridoma line and the antibody genes obtained by RT-PCR and cloned into an immunoglobulin expression vector. Alternatively, combinatorial immunoglobulin phagemid libraries are prepared from RNA isolated from the cell lines and phagemids expressing appropriate antibodies are selected by panning using viral antigens. The advantages of this approach over conventional hybridoma techniques are that approximately 10⁴ times as many antibodies can be produced and screened in a single round, and that new specificities are generated by H and L chain combination which further increases the chance of finding appropriate antibodies.

Other U.S. patents, each incorporated herein by reference, that teach the production of antibodies useful in the present invention include: U.S. Pat. No. 5,565,332, which describes the production of chimeric antibodies using a combinatorial approach; U.S. Pat. No. 4,816,567 which describes recombinant immunoglobulin preparations; U.S. Pat. No. 4,867,973 which describes antibody-therapeutic agent conjugates; and U.S. Pat. No. 8,563,305 which describes methods for rapidly producing monoclonal antibodies.

Included in this disclosure is a method of antibody generation from peripheral blood samples obtained from healthy donors immunized with two doses of the Pfizer (COMIRNATY®) vaccine. Also included in this disclosure is a method of antibody generation from peripheral blood samples collected from donors with history of COVID-19 infection who were later immunized with two doses of the Pfizer (COMIRNATY®) vaccine. The disclosure further includes the method wherein, blood samples were collected 7 days after the second vaccine dose followed by purification of peripheral blood mononuclear cells (PBMCs) and IgG-positive antibody-secreting cell isolation. One example of the method for generating human monoclonal antibodies from such samples is described below.

Lymphoprep and B-Cell Enrichment
1. Collect blood 7 days post-vaccination into acid citric dextrose blood collection tubes (typically one for ELISpot, four for sort, 40-50 ml of blood total).
2. Add RosetteSep at 2.5 μl/mL to whole blood. Mix well. Best results occur if the lymphoprep is begun immediately after collection of the blood but may be done within 18 h of collection. If necessary store blood overnight, store as whole blood at 4° C. and perform lymphoprep immediately prior to staining and cell sorting. The antibody secreting cells (ASC's) can become unstable and die when removed from whole blood and stored overnight or when frozen.
3. Incubate at room temperature (20-25° C.) for 20 min.
4. Dilute the blood with an equal volume of PBS.
5. Add 15 ml of LSM to a separate 50-ml conical tube. Carefully layer diluted blood over the LSM. Layer no more than 30 ml of diluted blood over 15 ml of LSM. Use multiple tubes if necessary.
6. Centrifuge for 30 min at 800 g at room temperature with no brake.
7. After centrifugation, the enriched PBMCs will form a band at the interface between the serum and the LSM. Remove this band with a Pasteur pipette and transfer to a new 50-ml centrifuge tube.
8. Rinse the enriched PBMCs by diluting to 50 ml with PBS, centrifuge for 5-10 min at 800 g at room temperature with no brake, then remove the supernatant.
9. If using more than one tube, combine the cells. Repeat Step 8, decreasing the centrifugation speed to 360 g. Brake may be used.

Staining and Flow Cytometry
10. To ensure that enough ASCs are obtained from the sorting process, begin with 4-8 million of enriched PBMCs prepared in Steps 1-9.
11. If the cells appear bloody (i.e., contain significant amounts of red blood cells), clear with ACK buffer (add 1 ml of ACK for 1-2 min). Wash the cells twice with PBS. Filter the cells through a 40-mm cell strainer to remove clumps.
12. Resuspend approximately 3 million cells in 100 μl of staining buffer; these are the cells that will be used for sorting. In addition, prepare one aliquot of cells (about 0.5×10 6 cells in 100 μl of staining buffer) for each fluorophore to be tested in Step 13 and one aliquot of cells that will remain unstained. These compensation controls will be used to adjust the sensitivity of the flow cytometer detectors to avoid overlap of the emission spectra when the various fluorophores are combined. All buffers for staining should contain 2% FCS (vol/vol) in PBS to block nonspecific staining.
13. Add the following antibodies to the aliquot of cells for sorting: CD3 FITC; anti-CD27 PE; anti-CD38 APC-Cy5.5; anti-CD20 FITC; anti-CD19 PE-Alexa Fluor 610; mouse anti-human IgM-biotin and anti-IgG-Alexa 647. In addition, add one of the fluorophore-conjugated antibodies to each of the compensation control aliquots of cells prepared in Step 12. The specific amounts of each antibody used should be titrated to give distinct single color populations before setting up a new experiment. Appropriate species-specific isotype control antibodies should be used to distinguish specifically stained populations from any background staining that might occur.
14. Incubate the cells for 30 min at 4° C.
15. Wash twice with 200 μl of 2% FCS in PBS.
16. Add 1:500 Streptavidin PE-Cy7 and incubate for 20 min at 4° C.
17. Wash twice again; pass the cells through another cell strainer to avoid clogs in the cytometer.
18. ASCs were gated as IgG+/CD19+/C D3−/CD20 low/CD27high/CD38high.
19. Bulk sort the cells into tubes containing 2% FCS in PBS buffer collecting the cells gated as above.
20. Re-sort the cells on forward versus side scatter (live cell gate with doublet discrimination) into single cell PCR plates containing 10 μl of RNase-inhibiting RT-PCR catch buffer. To facilitate the RT-PCR step, sort only into half of the plate and do not put cells in Row H (catch buffer should be added to this row to allow for PCR negative controls). Immediately seal each plate with a microseal foil label and place on dry ice until the cell sorting is finished when plates can be placed in a −80° C. freezer. Use RNase-free precautions for Step 20. As the catch buffer is hypotonic, the cells are lysed, and with immediate freezing, their RNA is protected by the included RNase inhibitor. It is necessary to use multiple buffer controls (row H) because the likelihood of PCR contamination increases substantially with the many cycles of PCR required to amplify the variable genes from single B cells. At this point, the plates may be stored for months to several years if they are immediately flash frozen on dry ice after the collection and kept at −80° C.

Reverse Transcription, Nested and Cloning PCRs
21. Thaw a half-plate of single cells on ice and prepare the RT-PCR master mix following the Bio-Rad iScript cDNA synthesis kit protocol. The program for the PCR machine is also as per the iScript protocol (also see Table 1). Each kit provides enough to process two half-plates. Add 10 μl of master mix to each well (now containing 20 μl of product). RNase-free precautions should be used for this step. When the program is complete, it is best to proceed with the next step (1st PCR, step 22), or immediately store the plate at −20° C. until ready to proceed. The protocol here only details the PCRs for IgG heavy chain and Kappa and Lambda light chain, as that is sufficient to amplify most antibodies of interest. However, there is enough master mix to do 5-6 subsequent reactions. These could include IgA and IgM heavy chains if not these are not well discriminated during the FACS sort and/or any other gene of interest (i.e., cytokines).
22. For the next PCR step (termed 1st PCR), prepare IgG heavy chain, kappa light chain, and lambda light chain master mixes (see Table 2 for master mixes and Table 3 for primer sequences for steps 22 and 23). Note that doing these simultaneously will require at least 2 PCR machines (the heavy chain program and light chain program are different, however the kappa and lambda mixes from each half plate can be combined onto a full plate and run simultaneously). Add 37 μl of IgG heavy master mix into 48 wells of a fresh 96 well plate. Add 3 μl of cDNA (per well) from step 21 to each well. Similarly add 37 μl of kappa light chain master mix and 3 μl of cDNA (per well) from step 21 and add 37 μl of lambda light chain master mix and 3 μl of cDNA (per well) from step 21. Remember that the 48 kappa and 48 lambda wells can be added to the same 96 well plate. Affix dome cap strip lids to the plate and place in the PCR machines. The PCR machine programs are listed in Table 1. After the program is complete, it is best to immediately run the 2nd PCR, if this is not possible, the plate can be stored at −20° C. until ready to proceed.

TABLE 1
cDNA Synthesis PCR Program
25° C. for 5 minutes
46° C. for 20 minutes
95° C. for 1 minute
4° C. hold
1st PCR IgG Heavy Chain Program
95° C. for 3 minutes
45 cycles of 95° C. for 30 seconds, 54° C. for 30 seconds, 68° C. for
1 minute
68° C. for 10 minutes
4° C. hold
1st PCR Kappa Light Chain Program (same for Lambda Light Chain)
95° C. for 3 minutes
45 cycles of 95° C. for 30 seconds, 55° C. for 30 seconds, 68° C. for
1 minute
68° C. for 10 minutes
4° C. hold
2nd PCR Program (same for IgG heavy, kappa light, and lambda
light chain)
95° C. for 3 minutes
40 cycles of 95° C. for 1 minute, 56° C. for 1 minute, 68° C. for 1 minute
68° C. for 10 minutes
4° C. hold

23. For the second PCR step with degenerate V-gene primers (termed 2nd PCR), again prepare IgG heavy chain, kappa light chain, and lambda light chain master mixes (see Table 2 for master mixes and Table 3 for primer sequences for steps 22 and 23). The 2nd PCR program is the same for all three combinations and thus if desired 48 heavy chain and 48 light chain wells can be combined onto the same plate and run in the same PCR machine. For all mixes, add 36 μl of master mix to each well. Then add 4 μl of template from the 1st PCR plate being careful to keep the 48 heavy chain wells and 48 light chain wells distinct. Affix dome cap strip lids to the plate and place in the PCR machines. The PCR machine programs are listed in Table 1.
24. When the 2nd PCR program is complete, make a 1% Agarose gel in TAE buffer (large enough for 96 wells). Run 1 μl from each well with the proper loading dye and marker. Positive wells have a band around 400 bp indicating successful amplification of an antibody gene. Wells positive for both a heavy and a light chain are noted for the following steps. We suggest proceeding with the PCR cleanup, but the plate can be frozen at −20° C. until ready to proceed if necessary.
25. Based on the number of positive wells, one of two methods can be used for the cleanup to prepare for sequencing. Although for a typical experiment, a large number (>70% of total single cells) are positive, occasionally with certain vaccines and certain vaccinees there may only be a few positive cells. If there are only a handful of positive wells, we suggest using a QIAquick PCR purification kit. If there are a large number of positives or you are processing a large number of plates simultaneously, we suggest an ‘entire plate’ ethanol precipitation as described here. Spin the second PCR plate on a swinging plate base at 800 rpm for 30 seconds. Add 75 in of freshly made ethanol/0.12M sodium acetate to each well. This solution is made with a stock solution of 3M sodium acetate at pH 4.5 (dissolve 408.24 g of sodium acetate trihydrate in about 600 ml of purified water, adjust the pH to 4.5 with glacial acetic acid and bring to a final volume of 1 L with purified water). Combine 15.7 ml of 100% ethanol, 660 in of 3M sodium acetate and 175 μl of purified water; this makes 16.5 ml, enough for two 96-well plates. Leaving the plate uncovered, spin immediately at 3200 rpm in a centrifuge pre-cooled to 4° C. for 35 minutes. Decant the ethanol/sodium acetate solution by placing the plate upside down on a paper towel in the swinging plate base, still cooled to 4° C., then allow the centrifuge to spin up to 900 rpm (about 5 seconds) and immediately stop the centrifuge. Flip the plate back over onto the benchtop. Rinse the plate by adding 70 μl of freshly prepared 70% ethanol, again centrifuging at 3200 rpm at 4° C. for 15 minutes, then flipping the plate onto a paper towel and spinning up to 900 rpm and stopping as above. Flip the plates over again, protect from dust with a paper towel and allow to air dry on the benchtop for an hour. Finally, resuspend the dried DNA is in 35 μl of sterile water per well. Wells corresponding to positive cells are transferred to 1.5 ml labeled Eppendorf tubes.
26. Sequence each purified well with conventional ABI/Sanger sequencing. The ‘reverse’ primer for the 2nd PCR is also used to sequence (nested rev, 3′ CK494-516, and CL_int_rev for heavy, kappa, and lambda respectively). After sequencing, the raw sequences are submitted to IMGT/Vquest with the output formatted for analysis and cataloging.
27. Prepare the cloning PCR master mixes as detailed in the Table 3. To ensure that the master mix is not contaminated, prepare each master mix with enough volume to have one buffer control. Many of the primers for the cloning PCR are used for several gene families as they prime conserved sequences. The targeted gene segments are all indicated in the name of the primer in Table 1. For example, the 5′ Agel VHl/5/7 primer is used for any gene from the VH1, VHS or VH7 families; the 5′ Agel VH3-9/30/33 primer is used for either VH3-9, VH3-30 65 or VH3-33 genes; the 3′ RSRII Jk 1/2/4 primer is used for either Jk 1, Jk 2 or Jk 4.
28. Add 1 μl of the RT product to each 24 μl of cloning PCR mix and apply dome caps as in Step 22. Products should be checked on a gel to ensure that a band is present and that the controls are not contaminated as described in Step 25. Run the PCR using the following conditions: 95° C. for 4 min, 35 cycles of 95° C. for 1 min, 57° C. for 1 min and 72° C. for 1.5 min.

TABLE 2

| 1st PCR IgG Heavy Chain Master Mix (7 primers) | 2nd PCR IgG Heavy Chain Master Mix (8 primers) |
|---|---|
| 1404.2 ul PCR Water | 1320.9 ul PCR Water |
| 200 ul 10X Taq Polymerase Buffer | 200 ul 10X Taq Polymerase Buffer |
| 16.7 ul IgG_ext_rev primer | 33.3 ul VH1/5/7_nested primer |
| 33.3 ul VH1/7_ext primer | 33.3 ul VH2_nested primer |
| 16.7 ul VH2_ext primer | 33.3 ul VH3_nested_A primer |
| 33.3 ul VH3_ext primer | 33.3 ul VH3_nested_B primer |
| 33.3 ul VH4_ext primer | 33.3 ul VH4_nested_A primer |
| 33.3 ul VH5_ext primer | 16.7 ul VH4_nested_B primer |
| 16.7 ul VH6_ext primer | 16.7 ul VH6_nested primer |
| 50 ul dNTP mix | 16.7 ul Nested_rev primer |
| 12.5 ul DNA Taq Polymerase Enzyme (standard Taq, 5000 U/ml) | 50 ul dNTP mix |
| Vtotal = 1850 ul (48 wells plus excess for pipet error) | 12.5 ul DNA Taq Polymerase Enzyme (standard Taq, 5000 U/ml) |
| | Vtotal = 1800 ul (48 wells plus excess for pipet error) |
| 1st PCR Kappa Light Chain Master Mix (6 primers) | 2nd PCR Kappa Light Chain Master Mix (6 primers) |
| 1454.1 ul PCR Water | 1387.5 ul PCR Water |
| 200 ul 10X Taq Polymerase Buffer | 200 ul 10X Taq Polymerase Buffer |
| 16.7 ul 3′CK 543 primer | 33.3 ul VK1_nested primer |
| 33.3 ul VK1_ext primer | 33.3 ul VK2_nested primer |
| 33.3 ul VK2_ext primer | 33.3 ul VK3_nested primer |
| 16.7 ul VK3_ext primer | 16.7 ul VK4_nested primer |
| 16.7 ul VK4_ext primer | 16.7 ul VK5_nested primer |
| 16.7 ul VK5_ext primer | 16.7 ul 3′ 494 nested primer |
| 50 ul dNTP mix | 50 ul dNTP mix |
| 12.5 ul DNA Taq Polymerase Enzyme (standard Taq, 5000 U/ml) | 12.5 ul DNA Taq Polymerase Enzyme (standard Taq, 5000 U/ml) |
| Vtotal = 1850 ul (48 wells plus excess for pipet error) | Vtotal = 1800 ul (48 wells plus excess for pipet error) |
| 1st PCR Lambda Light Chain Master Mix (9 primers) | 2nd PCR Lambda Light Chain Master Mix (using 8 primers) |
| 1337.6 ul PCR Water | 1271.1 ul PCR Water |
| 200 ul 10X Taq Polymerase Buffer | 200 ul 10X Taq Polymerase Buffer |
| 33.3 ul 3′CL_ext_rev primer | 33.3 ul 3′CL_int_rev primer |
| 33.3 ul 5′VL1/2_ext primer | 33.3 ul 5′VL1_int primer |
| 33.3 ul 5′VL3a_ext primer | 33.3 ul 5′VL2_int primer |
| 33.3 ul 5′VL3b_ext primer | 33.3 ul 5′VL3_int primer |
| 16.7 ul 5′VL4_ext primer | 33.3 ul 5′VL4_int primer |
| 16.7 ul 5′VL5_ext primer | 33.3 ul 5′VL5_int primer |
| 16.7 ul 5′VL7_ext primer | 33.3 ul 5′VL7/8_int primer |
| 33.3 ul 5′VL8_ext primer | 33.3 ul 5′VL9/10/11_int primer |
| 33.3 ul 5′VL9/10/11_ext primer | 50 ul dNTP mix |
| 50 ul dNTP mix | 12.5 ul DNA Taq Polymerase Enzyme (standard Taq, 5000 U/ml) |
| 12.5 ul DNA Taq Polymerase Enzyme (standard Taq, 5000 U/ml) | Vtotal = 1800 ul (48 wells plus excess for pipet error) |
| Vtotal = 1850 ul (48 wells plus excess for pipet error) | |

PCR Purification
29. Follow the protocol outlined in the QIAquick PCR Microcentrifuge Protocol with one exception: to elute the DNA, apply 31 μl of PCR water to the column, let the column sit for 1 min and then centrifuge. For all centrifuging steps, centrifuge for 60 s at 17,900 g (13,000 rpm) at room temperature as per Qiagen protocol. PCR products may be stored for up to 1 month at 20° C. First digestion of gamma, kappa or lambda chain variable gene inserts.
30. For all inserts: add 3.5 μl of NEB buffer 1 and 1 μl of Agel to purified PCR products (use manufacturer recommended appropriate buffers for the restriction system utilized).
31. Mix the sample by pipetting up and down.
32. Overlay the sample with 40 μl of sterile mineral oil.
33. Incubate the samples for 4 h or overnight in a 37° C. water bath or heat block.

Digestion Purification
34. Purify using the same protocol as the ‘PCR purification’ in Step 29.

Second Digestion
35. For a gamma chain insert, add 3.5 μl NEB buffer3, 0.35 μl BSA and 1 μl SalI to the purification product. For a kappa chain insert, add 3.5 μl NEB buffer 3 and 1 μl RSRII to the purification product. For a lambda chain insert, add 3.5 μl NEB buffer 2, 0.35 μl BSA and 1 μl XhoI to the purification product.
36. Overlay the sample with 40 μl of sterile mineral oil.
37. Incubate the sample for 4 h or overnight in a water bath. For kappa inserts, incubate at 55° C. For gamma and lambda inserts, incubate at 37° C.

Gel Purification
38. Run all samples on a 1% agarose gel (wt/vol). The insert band will be approximately 400 bp in length.
39. Follow the protocol outlined in the QIAquick Gel Extraction Kit (using a microcentrifuge) with one exception: to elute the DNA, apply 34 μl of EB buffer to the column, let the column sit for 1 min and centrifuge. Note: all centrifuge steps are carried out for 60 s. After excising the insert band from the gel, you may store it at 4° C. overnight before proceeding with the remaining gel purification protocol. The final product may be stored for up to 1 year at −20° C.

Ligation
40. Vector and insert DNA concentrations should be calculated from the A260 reading of a spectrophotometer (an A260 of 1.0 is 50 mg/ml of pure double stranded 20 DNA). A five-fold molar excess of insert to vector should be used. As the vector is approximately 5,700 bp and the insert is typically 350-400 by (variance is due to the CDR3 junction), a 3:1 ratio of vector to insert can be used.
41. Add 1 μl of vector (from a 1 μg/ml stock), 1 μl of T4 DNA ligase buffer, 1 μl of T4 ligase and an appropriate volume of the insert purification product to equal 0.3 mg into a clean 0.5 microcentrifuge tube.
42. Add PCR water to a final volume of 10 mL. Incubate the sample overnight in a bench-top temperature controlled mini-fridge or for 2 h at room temperature.

Transformation of DH5a Cells
43. Follow the protocol included with the DH5a cells with the following exceptions: use 25 μl of DH5a cells and 3 μl of DNA, and plate the cells on an LB plate containing 50 μg/ml of ampicillin. Incubate the cultures for 2-3 h in SOC media at 37° C., and plate 100 in of the transformation culture. Incubate the plates overnight at 37° C.
44. Choose four colonies from the plate to ensure a consensus variable gene sequence is identified. For each colony, inoculate one 14-ml round-bottom tube containing 5 ml of LB broth and ampicillin (50 μg/ml).
45. Incubate the tubes overnight, shaking at 225 r.p.m. on an orbital shaker, at 37° C.
46. Make glycerol stocks of each culture by transferring 300 ml of 1:1 sterile LB/glycerol and 700 ml of the confluent culture to a 2-ml tube, mix well and freeze at −80° C. These glycerol stocks are still viable after several years at −80° C.

Miniprep
47. Pellet bacteria by centrifuging the culture tubes (prepared in Steps 44 and 45) for 10 min at 800 g. Discard the supernatant.
48. Follow the protocol outlined in the QlAprep Spin Mini-55 prep Kit Handbook (using a microcentrifuge) with one exception: elute the DNA with 40 in of EB buffer. Note: all centrifuge steps are carried out for 60 s.
49. Sequence the eluted DNA with the AbVec primer.

Maxiprep
50. Compare the four mini-prep sequences using DNA sequence alignment software (Such as ClustalW: www.ebi.ac.uk/Tools/clustalw2/index.html). It is expected that some sequences will have accumulated base exchanges due to PCR errors but one of the four samples typically represents the consensus.
51. With a scraping from the glycerol stock of the colony of choice, inoculate one 14-ml round-bottom tube containing 5 ml of LB broth with ampicillin (50 μg/ml).
52. Incubate the tubes for 4-5 h, shaking at 225 rpm on an orbital shaker, at 37° C.
53. Transfer the cultures to 500-ml flasks containing 250 ml of LB broth and ampicillin (50 μg/ml). Incubate the flasks overnight, shaking at 225 rpm on an orbital shaker, at 37° C.
54. Follow the protocol outlined in the Genopure Plasmid Maxi Kit with the following exception: re-dissolve the plasmid DNA pellet in 400 μl of pre-warmed (50° C.) elution buffer.

Transfection of 293A Cells
55. 293A cells should be grown and passaged as per the product sheet from Invitrogen. Ensure that 293A cells are 80-90% confluent and evenly spread out across the 150 mm×25 mm tissue culture plate. It is important that the passage number for the 293A cells be kept below 30 passages; otherwise, the cells may not efficiently produce the antibody.
56. Warm DMEM media to room temperature; thaw PEI solution, heavy chain and light chain DNA.
57. For each plate to be transfected, aliquot 2.4 ml of DMEM into a conical vial. Add 9 μg of heavy chain DNA and 9 μg of light chain DNA per plate to the DMEM.
58. Add 100 ml of PEI solution per plate to the prepared DMEM and DNA mixture. Immediately vortex. Incubate at room temperature for 15 min.
59. Remove all but 18 ml of the culture media from each plate to be transfected.
60. Gently add 2.5 ml of PEI mixture to each plate, rocking the plate to ensure even distribution.
61. Incubate the cells with the PEI mixture in an incubator at 37° C. with 5% CO₂ for 24 h.
62. Change the culture media to basal media (20-25 ml per plate).
63. Collect the media from the plates 4 d later. The supernatant may be stored at 4° C. for several months if NaN₃ is added at a concentration of 0.05% (wt/vol). For some applications (i.e., ELISA), the antibody-containing supernatant is sufficient for testing the mAbs and the protein purification steps (Steps 64-77) can be optional. However, for long-term storage and more flexibility the antibodies are preferably purified.

Protein Purification
64. Prepare protein A agarose beads by adding approximately 1.5 ml of suspended beads to 50 ml of PBS in a 50-ml conical tube.
65. Centrifuge the tubes of beads for 10 min at 2,100 g at room temperature with no brake. Remove the PBS with an aspirator. Do not use brake on any of the centrifugations involving the agarose beads, as braking can damage the beads. Even slight breaking at the end of the spin can cause the beads to fluff, making it difficult to cleanly remove the supernatant.
66. Rinse each tube of beads with PBS (fill each tube with 50 ml of PBS and repeat Step 65).
67. Centrifuge the media collected from the transfection for 10 min at 900 g at room temperature, and then transfer the media from two plates (25 ml from each plate) to each tube of beads.
68. Incubate the media with the beads for 1-2 h at room temperature or overnight at 4° C. with slow agitation using a variable speed angle rocker. It works well to stabilize the tubes in a horizontal position.
69. Centrifuge the tubes of beads for 10 min at 2,100 g at room temperature with no brake. Remove the media with an aspirator.
70. Add 35 ml of 1 M NaCl to each tube. Centrifuge the tubes of beads for 10 min at 2,100 g at room temperature with no brake. Remove the 1 M NaCl with an aspirator.
71. Rinse each tube of beads with PBS (fill each tube with 35 ml of PBS and repeat Step 65).
72. Repeat Step 65.
73. Add 3-5 ml of 0.1 M glycine-HCl to each tube. Incubate on a tabletop shaker for 15 min.
74. Centrifuge the tubes of beads for 10 min at 2,100 g at room temperature with no brake. Transfer the glycine-HCl to a new vial. The time the antibodies are at low pH should be minimized as much as possible.
75. Adjust the pH to 7-7.4 with 1 M Tris-HCl. If there are beads in the vial, centrifuge the tubes for 10 min at 2,100 g at room temperature with no brake.
76. Transfer the neutralized sample to the top of an Amicon protein concentrator; add PBS to a final volume of 15 ml. Centrifuge the concentrator for 8-12 min at 2,100 g at room temperature with brake on, until a volume of 0.5-1.0 ml is reached.
77. Transfer the concentrated antibody sample from the concentrator into a clean 1.5-ml tube. If desired, preserve the antibody by adding NaN₃ to 0.05% (wt/vol). Note that biological assays using live cells (i.e., viral infection neutralization assays) are sensitive to NaN₃.
78. To reuse the beads (up to 10 times as suggested by the manufacturer), add 15 ml of 0.1 M glycine-HCl to each 30 tube of beads after 3-5 ml containing the antibody fraction is removed. Incubate on a tabletop shaker for 30 min, centrifuge for 10 min at 2,100 g at room temperature with no brake, remove the glycine-HCl with an aspirator, and then rinse twice with PBS (according to Step 89). Store in conical vials with 50 ml of PBS containing 0.05% NaN₃ at 4° C. for up to 6 months.

Protein Quantification
79. Follow the protocol included with the EZQ Protein Quantification Kit with the following exception: stain the paper for 60 min. Protein concentrations can be checked using an alternative quantification method, such as anti-IgG ELISA assays relative to a good IgG standard, the Qubit Protein Quantification Kit or a spectrophotometer. For critical applications, verify the concentrations by more than one method.

Gel Confirmation of Protein Quality
80. Run the resulting purified antibodies on an SDS-PAGE gel (12% gel (vol/vol), 4% stacking (vol/vol), reducing conditions). The resulting bands for heavy chain will be between 50 and 60 kDa and the light chain will be between 20 and 25 kDa.

Reagents for Steps 1-80

Ig gamma, Ig kappa and Ig lambda expression vectors: The expression vectors contain a murine immunoglobulin signal peptide sequence and variable-gene cloning sites upstream of the appropriate human immunoglobulin constant regions followed by an SV40 polyadenylation sequence. Transcription is under the HCMV (human cytomegalovirus immediate—early) promoter and clones are selected based on ampicillin resistance. The antibody variable-heavy and variable-light rearranged genes from each single cell are cloned into the respective vectors in frame with the signal peptide and constant region genes. These vectors are then co-transfected into the 293A cell line for expression. The resultant antibodies are properly trafficked and secreted after cleavage of the signal peptide, resulting in fully human IgG/kappa or IgG/lambda amino acid sequences. The vector sequences are available through the NCBI GenBank (accession numbers: FJ475055, FJ475056 and FJ517647).

Basal media: An aliquot of 250 ml each of sterile RPMI and DMEM; 3.75 ml of antibiotic/antimycotic and 5 ml each of L-glutamine (200 mM), 100× Nutridoma and sodium pyruvate (100 mM) was used. Basal media must be made fresh every 7 d. L-Glutamine can be stored at −20° C. for up to 1 year, Nutridoma can be stored at room temperature (20-25° C.) for up to 1 year and sodium pyruvate can be stored for up to 6 months at 4° C.

0.1 M glycine-HCl: Here 0.1 M glycine solution is equilibrated to pH 2.7 with 12 M HCl and filter sterilized. Solution can be stored up to 60 d at room temperature.

1M Tris-HCl: Here 1M Tris solution is equilibrated to pH 9.0 with HCl and filter sterilized. Solution can be stored up to 60 d at 4° C.

ACK lysing buffer: Here 0.15 M NH₄Cl, 10 mM KHCO₃ and 0.1 mM Na₂ EDTA. Adjust pH to 7.2-7.4 with 1M HCl and filter sterilized. Solution can be stored up to 1 year at room temperature (20-25° C.).

LB agar plates: LB agar dissolved in dH2O according to package directions and autoclaved. When cooled to 45° C., 50 μg/ml ampicillin is added. Dispense 20-25 ml agar solution into 100 mm×15 mm petri dishes. Cool and store at 4° C. for up to 6 months.

AEC substrate: Prepare AEC stock (20 mg/ml AEC in dimethylformamide). Dilute AEC from stock to 0.3 mg/ml in 0.1 M sodium acetate buffer (pH 5.0) just prior to use. Filter sterilized with a syringe filter. The stock solution may be made and stored for up to 2 months. The diluted solution must be made fresh each time used.

RNAse-inhibiting RT-PCR catch buffer: To 5 ml of RNAse-free water, add 50 μl of 1M Tris pH 8.0 and 125 μl of Rnasin. Keep on ice. This makes enough for 10 half plates. Catch buffer must be made fresh each time used.

PEI solution: prepare a 1 mg/ml PEI solution in 100 mL dH2O. Heat to 80° C. (do not boil) to allow all of the PEI to dissolve, then allow to cool. Adjust pH to 7.2 with HCl. Filter sterilize with a syringe filter. Store aliquots at −20° C. for up to 1 year.

TABLE 3
SEQ ID NOS: 25-71.

| 1^{st} PCR | | 2^{nd} PCR | |
|---|---|---|---|
| VH1/7_ext | ATGGACTGGACCTGGAGS | VH1/5/7_nested | SAGGT/dl/CAGCTGGTGCARTC |
| VH2_ext | CATACTTTGTTCCACGCTCCTG | VH2_nested | CAGRTCACCTTGARGGAGTCTGGTC |
| VH3_ext | AGGTGTCCAGTGTSARGTGC | VH3_nested_A | SAGGTGCAGCTGGTGGAGTC |
| VH4_ext | GTGGCRGCTCCCAGATG | VH3_nested_B | GARGTGCAGCTGKTGGAGTC |
| VH5_ext | GTTCTCCAAGGAGTCTGTKCCG | VH4_nested_A | CAGSTGCAGCTGCAGGAGTC |
| VH6_ext | CTGTCTCCTTCCTCATCTTCCTG | VH4_nested_B | CAGGTGCAGCTACAGCAGTGG |
| IgG_ext_rev | TCTTGTCCACCTTGGTGTTGC | VH6_nested | CAGGTACAGCTGCAGCAGTCAG |
| VK1_ext | GAGGGTCCYYGCTCAGCTCCTG | Nested_rev | GTCCTTGACCAGGCAGCCCAG |
| VK2_ext | GAGGCTCCYTGCTCAGCTYCTG | VK1_nexted | CATCCAGWTGACCCAGTCTCCATC |
| VK3_ext | CTCTTCCTCCTGCTACTCTGGCTC | VK2_nested | TTGTGATGACYCAGWCTCCACTC |
| VK4_ext | GTGTTGCAGACCCAGGTCTTCATTTC | VK3_nested | CAGTCTCCAGSCACCCTG |
| VK5_ext | GTTCACCTCCTCAGCTTCCTCCTC | VK4_nested | CATCGTGATGACCCAGTCTCCAG |
| 3′ CK 543-566 | GTTTCTCGTAGTCTGCTTTGCTCA | VK5_nested | GAAACGACACTCACGCAGTCTC |
| VL1/2_ext | GRCACAGG/dl/TCYTGGGC | 3′ CK 494-516 | GTGCTGTCCTTGCTGTCCTGCT |
| VL3a_ext | CAGKCTCTG/dl/GRCCTCC | VL1_int | CAGTCTGTS/dl/TGACGCAGC |
| VL3b_ext | CAGG/dl/TCYGTGGCCTCC | VL2_int | CAGTCTGCCCTGAYTCAGCC |
| VL4_ext | CCACTGCACAGGGTCTCTCTC | VL3_int | CCTCCTATGWGCTGACWCAGCC |
| VL5_ext | CTCACTGCACAGGTTCCCTCTC | VL4_int | CCCAGCYTGTGCTGACTCAATC |
| VL7_ext | CACTTGCTGCCCAGGGTC | VL5_int | CAGSCTGTGCTGACTCAGC |
| VL8_ext | CTCCTTGCTTATGGRTCAGGRGTG | VL7/8_int | CAG/dl/CTGTGGTGACYCAGG |
| VL9/10/11_ext | CCAGSC/dl/GKGCTGAC | VL9/10/11_int | GCTGACTCAGCCRCCYTC |
| CL_ext_rev | GACGGGGCTGCYATCTGC | CL_int_rev | CYAGTGTGGCCTTGTTGGCTTG |
| Additional cPCR primers | | | |
| 3′RSRII_JK1/2/4 | GCCACGGTCCGTTTGATYTCCACCTTGGTC | | |
| 3′RSRII_JK3 | GCCACGGTCCGTTTGATATCCACTTTGGTC | | |
| 3′RSRII_JK5 | GCCACGGTCCGTITAATCTCCAGTCGTGTC | | |

K G or T
R A or G
S G or C
W A or T
Y C or T
/di/ deoxyinosine

To test the binding specificity and affinity of the antibodies p2C01 and p3C03, binding curves were generated as described herein. Wells were coated with 200 ng of Wuhan or variant 51 or RBD blocked with 0.1% BSA in PBS, and developed with anti-human IgG-HRP (Jackson ImmunoResearch, West Grove, PA) and Super Aqua Blue substrate (EBiosciences, San Diego CA). The absorbance was measured at 405 nm on a microplate reader (Molecular Devices, Sunnyvale, CA). Antibody affinities (Kd) were calculated by curve fitting analysis of individual ELISA curves plotted from a dilution series of 16 two-fold dilutions of antibody beginning at 10 μg/ml. Binding curves were generated with a saturation binding, non-linear curve fit using GraphPad Prism software. Equilibrium dissociation constants (Kd) values for each hmAb were calculated using the equation Y=Bmax*X/(Kd+X) where Bmax is the maximum number of binding sites, X is the concentration of the antibody and Y is the specific binding. Therefore, the reported dissociation constants are equal to the concentration of antibody where half the binding sites are occupied at equilibrium. Each antibody was run in duplicate in at least three unique experiments. The binding of Ab to various SARS-CoV-2 strains such as Wuhan, Epsilon (B.1.427 and B.1.429), Gamma (P.1), Alpha (B1.1.7), Beta (B.1.351), Delta (B.1.617.2.1) and Omicron (B.1.1.529) and SARS-CoV was examined. The results for each experiment were then averaged to obtain the reported Kd in Table 4.

TABLE 4

Affinity

| Strain | p2C01 | p3C03 |
|---|---|---|
| Wuhan RBD_avg | 0.36 nM | 0.14 nM |
| Wuhan S1_avg | 0.09 nM | 0.51 nM |
| B.1.427_avg | 0.13 nM | 0.16 nM |
| B.1.429_avg | 0.10 nM | 0.12 nM |
| P.1_avg | 0.10 nM | 0.19 nM |
| B.1.1.7_avg | 0.15 nM | 0.19 nM |
| B.1.351_avg | 0.09 nM | 0.20 nM |
| B.1.617.2.1_avg | 0.10 nM | 0.13 nM |
| B.1.1.529 RBD_avg | 0.19 nM | 0.57 nM |
| SARS1-RBD_avg | NB | 0.34 nM |

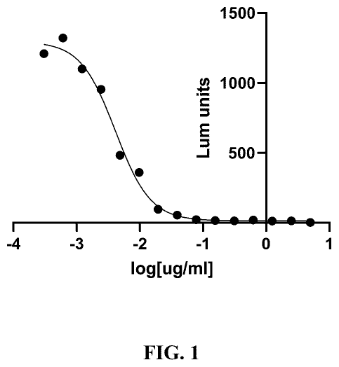

To test the neutralization capacity of the antibodies generated, pseudovirus/lentiviral neutralization assays were conducted using commercial S-pseudotyped lentivirus (BPS Biosciences) in a 96-well format. The virus was incubated with the monoclonal antibodies samples of interest (15 two-fold dilutions) for an hour at 37° C. The virus/antibody mixture was then added to HEK-293T-hACE2 cells for 60-72 hours. Luminescence was measured using the One-Step Luciferase Assay System (BPS Biosciences) on a luminescence plate reader. Each sample was run in duplicate, the values for each dilution were averaged and IC50's determined using a 4-parameter/sigmoidal curve fit (FIG. 1).

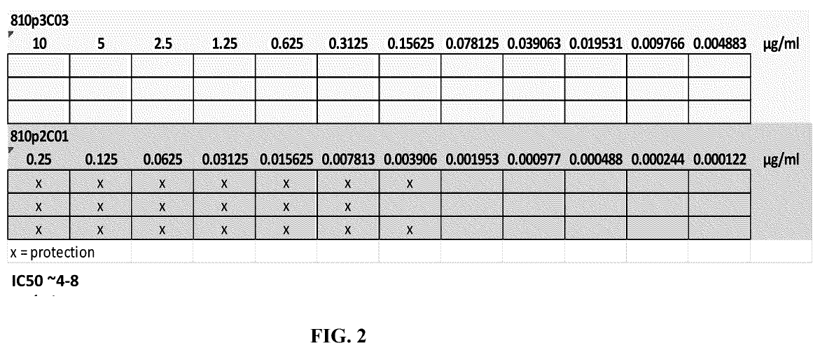

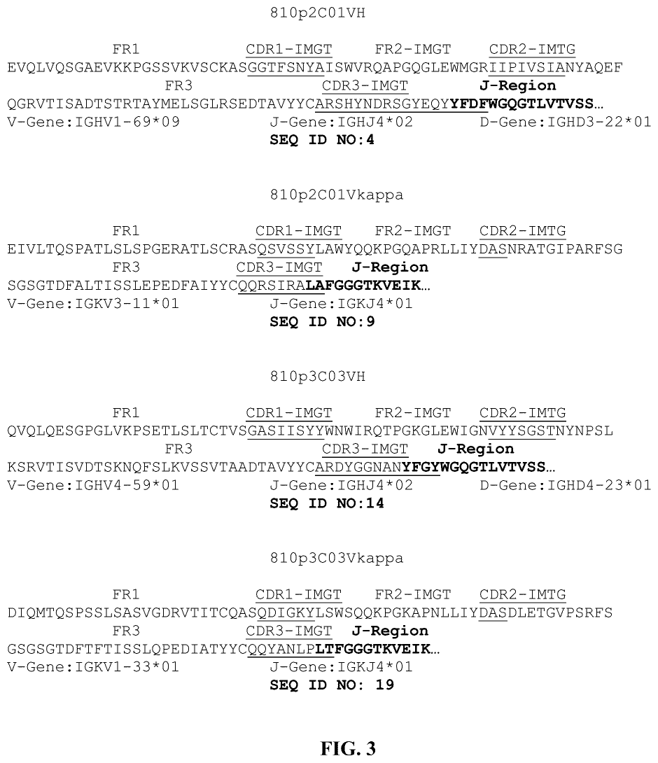

To further assess SARS-CoV-2 neutralization, each antibody was serially diluted 1:2 across a 96-well plate and mixed with enough virus (isolate USA-WA1/2020) to yield a final MOI of 0.01. After incubating the antibody virus mixture for 1 h at 37° C., the virus mixture was transferred to a 96-well plate containing VERO E6 cells seeded at 10,000 cells per well. SARS-CoV-2 activity was then determined 96 h after infection by visually observing cytopathic effects (CPE). The antibody dilution at which virus positive wells was observed was recorded as shown in FIG. 2. The V(D)J gene usage and protein sequences for Abs 810p2C01 and 810p3CO3 are described in FIG. 3.

FIG. 3 shows the amino acid sequence for the framework regions (FR) 1-3, CDR 1-3 and joining (J) regions of the heavy and light chains of antibodies designated 810p2C01 and 810p3C03 that have SARS-CoV-2 binding capacity. The figure also provides the genes utilized for the variable (V), diversity (D) and joining (J) regions of the heavy chains and the genes utilized in the V and J region of the light chains of the two antibodies. SEQ ID NOS:4, 9, 14, and 19, are as indicated in the figure, with the framework regions and CDRs labeled, as shown.

The 810p2C01 heavy and light chain nucleotide and protein sequences are as follows:

810p2C01VH:
(SEQ ID NO: 21)
gaggtgcagctggtgcagtctggggctgaggtgaagaagcctgggtcctc
ggtgaaggtatcctgcaaggcttctggaggcaccttcagcaactatgcta
tcagctgggtgcgacaggccccgggacaagggcttgagtggatgggaagg
atcatccccatcgttagtatagcaaactacgcacaggagtttcagggcag
agtcacgattagcgcggacacatccacgcgcacagcctatatggaactca
gcggcctgagatctgaggacacggccgtgtattactgtgcgaggtcgcat
tacaatgataggagtggttatgaacaatattactttgacttctggggcca
gggaaccctggtcaccgtctcctcag.
810p2C01VH:
(SEQ ID NO: 4)
EVQLVQSGAEVKKPGSSVKVSCKASGGTFSNYAISWVRQAPGQGLEWMGR
IIPIVSIANYAQEFQGRVTISADTSTRTAYMELSGLRSEDTAVYYCARSH
YNDRSGYEQYYFDFWGQGTLVTVSS.
810p2C01Vkappa:
(SEQ ID NO: 22)
gaaattgtgttgacacagtctccagccaccctgtctttgtctccagggga
aagagccaccctctcctgcagggccagtcagagtgttagcagctacttag
cctggtaccaacagaaacctggccaggctcccaggctcctcatctatgat
gcatccaacagggccactggcatcccagccaggttcagtggcagtgggtc
tgggacagacttcgctctcaccatcagcagcctagagcctgaagactttg
caatttattactgtcagcaacgtagcatccgggcgctcgctttcggcgga
gggaccaaggtggaaatcaaac.
810p2C01Vkappa:
(SEQ ID NO: 9)
EIVLTQSPATLSLSPGERATLSCRASQSVSSYLAWYQQKPGQAPRLLIYD
ASNRATGIPARFSGSGSGTDFALTISSLEPEDFAIYYCQQRSIRALAFGG
GTKVEIK.

The 810p3C03 heavy and light chain protein and nucleotide sequences are as follows:

810p3C03VH:
(SEQ ID NO: 23)
caggtgcagctgcaggagtcgggcccaggactggtgaagccttcggagac
cctgtccctcacctgcactgtatctggtgcctccatcattagttactact
ggaactggatccggcagaccccagggaagggactggagtggattgggaat
gtctattacagtgggagcaccaactacaacccctccctcaagagtcgagt
caccatatcagtagacacgtccaagaaccagttctccctgaaggtgagct
ctgtgaccgctgcggacacggccgtctattactgtgcgagagactacggt
ggtaacgcgaactactttgggtactggggccagggaaccctggtcaccgt
ctcctcag.
810p3C03VH:
(SEQ ID NO: 14)
QVQLQESGPGLVKPSETLSLTCTVSGASIISYYWNWIRQTPGKGLEWIGN
VYYSGSTNYNPSLKSRVTISVDTSKNQFSLKVSSVTAADTAVYYCARDYG
GNANYFGYWGQGTLVTVSS.
810p3C03Vkappa:
(SEQ ID NO: 24)
gacatccagatgacccagtctccatcctccctgtctgcatctgtaggaga
cagagtcaccatcacttgccaggcgagtcaggacattggcaagtatttaa
gttggtctcagcagaaaccagggaaagcccctaacctcctgatctacgat
gcatccgatttggaaacaggggtcccatctaggttcagtggaagtggatc
tgggacagattttactttcaccatcagcagcctgcagcccgaagatattg
caacatattactgtcaacagtatgctaatctcccgctcactttcggcgga
gggaccaaggtggagatcaaac.
810p3C03Vkappa:
(SEQ ID NO: 19)
DIQMTQSPSSLSASVGDRVTITCQASQDIGKYLSWSQQKPGKAPNLLIYD
ASDLETGVPSRFSGSGSGTDFTFTISSLQPEDIATYYCQQYANLPLTFGG
GTKVEIK.

Additional oligonucleotides for use with the invention are listed as SEQ ID NOS: 73-100, which include the restriction enzyme cleavage site and the location on the antibody, as follows:

| | SEQ ID | | |
|---|---|---|---|
| Primer | NO: | Sequence | |
| 5′ AgeI VH3 | 73 | CTGCAACCGGTGTACATTCTGAGGTGCAGCTG | Cloning |
| | | GTGGAG | PCR |
| 5′ AgeI VH3-23 | 74 | CTGCAACCGGTGTACATTCTGAGGTGCAGCTG | Cloning |
| | | TTGGAG | PCR |
| 5′ AgeI VH4 | 75 | CTGCAACCGGTGTACATTCCCAGGTGCAGCTG | Cloning |
| | | CAGGAG | PCR |
| 5′ AgeI VH4-34 | 76 | CTGCAACCGGTGTACATTCCCAGGTGCAGCTA | Cloning |
| | | CAGCAGTG | PCR |
| 5′ AgeI VH1-18 | 77 | CTGCAACCGGTGTACATTCCCAGGTTCAGCTG | Cloning |
| | | GTGCAG | PCR |
| 5′ AgeI VH1-24 | 78 | CTGCAACCGGTGTACATTCCCAGGTCCAGCTG | Cloning |
| | | GTACAG | PCR |
| 5′ AgeI VH3- | 79 | CTGCAACCGGTGTACATTCTGAAGTGCAGCTG | Cloning |
| 9/30/33 | | GTGGAG | PCR |
| 5′ AgeI VH6-1 | 80 | CTGCAACCGGTGTACATTCCCAGGTACAGCTG | Cloning |
| | | CAGCAG | PCR |
| 5′ AgeI VK1 | 81 | CTGCAACCGGTGTACATTCTGACATCCAGATG | Cloning |
| | | ACCCAGTC | PCR |
| 5′ AgeI VK1- | 82 | TTGTGCTGCAACCGGTGTACATTCAGACATCC | Cloning |
| 9/1-13 | | AGTTGACCCAGTCT | PCR |
| 5′ AgeI VK1D- | 83 | CTGCAACCGGTGTACATTGTGCCATCCGGATG | Cloning |
| 43/1-8 | | ACCCAGTC | PCR |
| 5′ AgeI VK2 | 84 | CTGCAACCGGTGTACATGGGGATATTGTGATG | Cloning |
| | | ACCCAGAC | PCR |
| 5′ AgeI VK2- | 85 | CTGCAACCGGTGTACATGGGGATATTGTGATG | Cloning |
| 28/2-30 | | ACTCAGTC | PCR |
| 5′ AgeI VK3- | 86 | TTGTGCTGCAACCGGTGTACATTCAGAAATTGT | Cloning |
| 11/3D-11 | | GTTGACACAGTC | PCR |
| 5′ AgeI VK3- | 87 | CTGCAACCGGTGTACATTCAGAAATAGTGATG | Cloning |
| 15/3D-15 | | ACGCAGTC | PCR |
| 5′ AgeI VK3- | 88 | TTGTGCTGCAACCGGTGTACATTCAGAAATTGT | Cloning |
| 20/3D-20 | | GTTGACGCAGTCT | PCR |
| 5′ AgeI VK4-1 | 89 | CTGCAACCGGTGTACATTCGGACATCGTGATG | Cloning |
| | | ACCCAGTC | PCR |
| 5′ AgeI VL1 | 90 | CTGCTACCGGTTCCTGGGCCCAGTCTGTGCTGA | Cloning |
| | | CKCAG | PCR |
| 5′ AgeI VL2 | 91 | CTGCTACCGGTTCCTGGGCCCAGTCTGCCCTGA | Cloning |
| | | CTCAG | PCR |
| 5′ AgeI VL3 | 92 | CTGCTACCGGTTCTGTGACCTCCTATGAGCTGA | Cloning |
| | | CWCAG | PCR |
| 5′ AgeI VL4/5 | 93 | CTGCTACCGGTTCTCTCTCSCAGCYTGTGCTGA | Cloning |
| | | CTCA | PCR |
| 5′ AgeI VL6 | 94 | CTGCTACCGGTTCTTGGGCCAATTTTATGCTGA | Cloning |
| | | CTCAG | PCR |
| 5′ AgeI VL7/8 | 95 | CTGCTACCGGTTCCAATTCYCAGRCTGTGGTGA | Cloning |
| | | CYCAG | PCR |
| 3′ SalI JH1/2/4/5 | 96 | TGCGAAGTCGACGCTGAGGAGACGGTGACCAG | Cloning |
| | | | PCR |
| 3′ SalI JH3 | 97 | TGCGAAGTCGACGCTGAAGAGACGGTGACCAT | Cloning |
| | | TG | PCR |
| 3′ SalI JH6 | 98 | TGCGAAGTCGACGCTGAGGAGACGGTGACCGT | Cloning |
| | | G | PCR |
| 3′ XhoI C1 | 99 | CTCCTCACTCGAGGGYGGGAACAGAGTG | Cloning |
| | | | PCR |
| AbVec | 100 | GCTTCGTTAGAACGCGGCTAC | Sequencing |

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 12 or 15%.

Additionally, the section headings herein are provided for consistency with the suggestions under 37 CFR 1.77 or otherwise to provide organizational cues. These headings shall not limit or characterize the invention(s) set out in any claims that may issue from this disclosure. Specifically and by way of example, although the headings refer to a “Field of Invention,” such claims should not be limited by the language under this heading to describe the so-called technical field. Further, a description of technology in the “Background of the Invention” section is not to be construed as an admission that technology is prior art to any invention(s) in this disclosure. Neither is the “Summary” to be considered a characterization of the invention(s) set forth in issued claims. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of such claims shall be considered on their own merits in light of this disclosure, but should not be constrained by the headings set forth herein.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

For each of the claims, each dependent claim can depend both from the independent claim and from each of the prior dependent claims for each and every claim so long as the prior claim provides a proper antecedent basis for a claim term or element.

### REFERENCES

1. Munster V. J. A novel coronavirus emerging in China-key questions for impact assessment. N. Engl. J. Med. 2020; 382(8):692-694.
2. Liu D. X., Liang J. Q., Fung T. S. Reference Module in Life Sciences. 2020. Human Coronavirus-229E, -OC43, -NL63, and -HKU1. p. B978-O-12-809633-8.21501-X.
3. Azhar E. I. Evidence for camel-to-human transmission of MERS coronavirus. N. Engl. J. Med. 2014; 370(26):2499-2505.
4. Guan Y. Isolation and characterization of viruses related to the SARS coronavirus from animals in southern China. Science. 2003; 302(5643):276-278.
5. Zhang Y.-Z., Holmes E. C. A genomic perspective on the origin and emergence of SARS-CoV-2. Cell. 2020; 181(2):223-227.
6. Kuba, K. et al. A crucial role of angiotensin converting enzyme 2 (ACE2) in SARS coronavirus-induced lung injury. Nat. Med. 11, 875-879 (2005).
7. Tang, T., Bidon, M., Jaimes, J. A., Whittaker, G. R. & Daniel, S. Coronavirus membrane fusion mechanism offers a potential target for antiviral development. Antivir. Res. 178, 104792 (2020).
8. Whittaker, G., Daniel, S. & Millet, J. Coronavirus entry: how we arrived at SARS-CoV-2. Curr. Opin. Virol. 47, 113-120 (2021).
9. Moss P. The T cell immune response against SARS-CoV-2. Nat Immunol. 2022 February; 23(2):186-193. doi: 10.1038/s41590-021-01122-w. Epub 2022 Feb. 1. PMID: 35105982.
10. It R√∂ltgen K, Boyd S D. Antibody and B cell responses to SARS-CoV-2 infection and vaccination. Cell Host Microbe. 2021 Jul. 14; 29(7):1063-1075. doi: 10.1016/j.chom.2021.06.009. Epub 2021 Jun. 17. PMID: 34174992; PMCID: PMC8233571.
11. Wang J, Jiang M, Chen X, Montaner L J. Cytokine storm and leukocyte changes in mild versus severe SARS-CoV-2 infection: Review of 3939 COVID-19 patients in China and emerging pathogenesis and therapy concepts. J Leukoc Biol. 2020 July; 108(1):17-41.
12. Azkur A K, Akdis M, Azkur D, Sokolowska M, van de Veen W, Br g′ggen MC, O'Mahony L, Gao Y, Nadeau K, Akdis C A. Immune response to SARS-CoV-2 and mechanisms of immunopathological changes in COVID-19. Allergy. 2020 July; 75(7):1564-1581.
13. Conti P, Ronconi G, Caraffa A, Gallenga C E, Ross R, Frydas I, Kritas S K. Induction of pro-inflammatory cytokines (IL-1 and IL-6) and lung inflammation by Coronavirus-19 (COVI-19 or SARS-CoV-2): anti-inflammatory strategies. 2020 March-April, J Biol Regul Homeost Agents. 34(2):327-331.
14. Coopersmith C M, Antonelli M, Bauer S R, Deutschman C S, Evans L E, Ferrer R, Hellman J, Jog S, Kesecioglu J, Kissoon N, Martin-Loeches I, Nunnally M E, Prescott H C, Rhodes A, Talmor D, Tissieres P, De Backer D. The Surviving Sepsis Campaign: Research Priorities for Coronavirus Disease 2019 in Critical Illness. Crit Care Med. 2021 Apr. 1; 49(4):598-622.
15. Lauer S A, Grantz K H, Bi Q, Jones F K, Zheng Q, Meredith H R, Azman A S, Reich N G, Lessler J. The Incubation Period of Coronavirus Disease 2019 (COVID-19) From Publicly Reported Confirmed Cases: Estimation and Application. Ann Intern Med. 2020 May 5; 172(9):577-582.
16. Thomson E C, Rosen L E, Shepherd J G, Spreafico R, da Silva Filipe A, Wojcechowskyj J A, Davis C, Piccoli L, Pascall D J, Dillen J, Lytras S, Czudnochowski N, Shah R, Meury M, Jesudason N, De Marco A, Li K, Bassi J, O'Toole A, Pinto D, Colquhoun R M, Culap K, Jackson B, Zatta F, Rambaut A, Jaconi S, Sreenu V B, Nix J, Zhang I, Jarrett R F, Glass W G, Beltramello M, Nomikou K, Pizzuto M, Tong L, Cameroni E, Croll T I, Johnson N, Di Iulio J, Wickenhagen A, Ceschi A, Harbison A M, Mair D, Ferrari P, Smollett K, Sallusto F, Carmichael S, Garzoni C, Nichols J, Galli M, Hughes J, Riva A, Ho A, Schiuma M, Semple M G, Openshaw P J M, Fadda E, Baillie J K, Chodera J D; ISARIC4C Investigators; COVID-19 Genomics UK (COG-UK) Consortium, Rihn S J, Lycett S J, Virgin H W, Telenti A, Corti D, Robertson D L, Snell G. Circulating SARS-CoV-2 spike N439K variants maintain fitness while evading antibody-mediated immunity. Cell. 2021 Mar. 4; 184(5):1171-1187.e20. doi: 10.1016/j.cell.2021.01.037. Epub 2021 Jan. 28. PMID: 33621484; PMCID: PMC7843029.
17. Houriiyah Tegally, Eduan Wilkinson, Marta Giovanetti, Arash Iranzadeh, Vagner Fonseca, Jennifer Giandhari, Deelan Doolabh, Sureshnee Pillay, Emmanuel James San, Nokukhanya Msomi, Koleka Mlisana, Anne von Gottberg, Sibongile Walaza, Mushal Allam, Arshad Ismail, Thabo Mohale, Allison J Glass, Susan Engelbrecht, Gert Van Zyl, Wolfgang Preiser, Francesco Petruccione, Alex Sigal, Diana Hardie, Gert Marais, Marvin Hsiao, Stephen Korsman, Mary-Ann Davies, Lynn Tyers, Innocent Mudau, Denis York, Caroline Maslo, Dominique Goedhals, Shareef Abrahams, Oluwakemi Laguda-Akingba, Arghavan Alisoltani-Dehkordi, Adam Godzik, Constantinos Kurt Wibmer, Bryan Trevor Sewell, Jos√© Louren√ßo, Luiz Carlos Junior Alcantara, Sergei L Kosakovsky Pond, Steven Weaver, Darren Martin, Richard J Lessells, Jinal N Bhiman, Carolyn Williamson, Tulio de Oliveira; medRxiv 2020.12.21.20248640; doi: https://doi.org/10.1101/2020.12.21.20248640.
18. Leung K, Shum M H, Leung G M, Lam T T, Wu J T. Early transmissibility assessment of the N501Y mutant strains of SARS-CoV-2 in the United Kingdom, October to November 2020. Euro Surveill. 2021 January; 26(1):2002106. doi: 10.2807/1560-7917.ES 0.2020.26.1.2002106. Erratum in: Euro Surveill. 2021 January; 26(3): PMID: 33413740; PMCID: PMC7791602.
19. Rambaut, A., Holmes, E. C., O'Toole, I A. et al. A dynamic nomenclature proposal for SARS-CoV-2 lineages to assist genomic epidemiology. Nat Microbiol 5, 1403-1407 (2020). https://doi.org/10.1038/s41564-020-0770-5.
20. CDC COVID-19 Response Team. SARS-CoV-2 B.1.1.529 (Omicron) Variant—United States, Dec. 1-8, 2021. MMWR Morb Mortal Wkly Rep. 2021 Dec. 17; 70(50):1731-1734. doi: 10.15585/mmwr.mm7050e1. PMID: 34914670; PMCID: PMC8675659.

1. A method for treating a glioblastoma in a human subject consisting essentially:
identifying the human subject in need of treatment of a glioblastoma, wherein the human is no longer responsive to at least one of chemotherapy, surgery, or radiation therapy as a result of increased ceramide production in the glioblastoma cell; and
administering to the human subject no longer responsive to at least one of chemotherapy, surgery, or radiation therapy, a therapeutically effective amount of a composition consisting of:
an amount of a curcumin or curcuminoids in one or more liposomes, or curcumin or curcuminoids and empty liposomes administered prior to, concomitantly, or after administration of the curcumin or curcuminoids, that is effective for treating the glioblastoma, wherein the liposomal curcumin or curcuminoids, or empty liposomes, eliminate the QT prolongation caused by the curcumin or curcuminoids; and
at least one chemotherapeutic agent, wherein the amount of the curcumin or curcuminoids is synergistic with the at least one chemotherapeutic agent to treat the glioblastoma, wherein the at least one chemotherapeutic agent is selected from at least one of etoposide, carmustine, lomustine, ceramide and phosphorylcholine that is no longer responsive to at least one of chemotherapy, surgery, or radiation therapy, wherein the glioblastoma cells are sensitized to an agent to which they have become refractory as a result of the increased ceramide production in the glioblastoma cell.

2. (canceled)

3. The method of claim 1, wherein the QT prolongation is LQTS.

4. The method of claim 3, wherein the liposomal curcumin or curcuminoids, or empty liposomes are provided intravenously.

5. The method of claim 1, wherein the composition increases ceramide production of the glioblastoma cell.

6. The method of claim 1, wherein the composition increases phosphorylcholine production of the glioblastoma cell.

7. The method of claim 1, wherein the glioblastoma cells are sensitized to an agent to which they have become refractory as a result of increased ceramide production in the glioblastoma cell.

8. The method of claim 1, wherein the glioblastoma cells are sensitized to an agent to which they have become refractory as a result of increased phosphorylcholine production in the glioblastoma cell.

9. A method for treating a glioblastoma in a human subject comprising the steps of:
identifying the human subject in need of treatment of a glioblastoma, wherein the human is no longer responsive to at least one of chemotherapy, surgery, or radiation therapy as a result of increased ceramide production in the glioblastoma cell; and
concurrently administering to the human subject no longer responsive to at least one of chemotherapy, surgery, or radiation therapy, a therapeutically effective amount of a composition consisting of:
an amount of a curcumin or curcuminoids in one or more liposomes, or curcumin or curcuminoids and empty liposomes;
an amount of one or more liposomes or empty liposomes prior to, concomitantly, or after administration of the curcumin or curcuminoids, that is effective for treating the glioblastoma, wherein the liposomal curcumin or curcuminoids, or the empty liposomes that eliminate the QT prolongation caused by the curcumin or curcuminoids; and
administering at least one chemotherapeutic agent, wherein the amount of the curcumin or curcuminoids is synergistic with the at least one chemotherapeutic agent to treat the glioblastoma, selected from at least one of etoposide, carmustine, lomustine, ceramide and phosphorylcholine to treat the glioblastoma that is no longer responsive to at least one of chemotherapy, surgery, or radiation therapy, wherein the glioblastoma cells are sensitized to an agent to which they have become refractory as a result of the increased ceramide production in the glioblastoma cell.

10. The method of claim 9, wherein the QT prolongation is LQTS.

11. The method of claim 9, wherein the liposomal curcumin or curcuminoids, or empty liposomes are provided intravenously.

12. The method of claim 9, wherein the composition increases ceramide production of the glioblastoma cell.

13. The method of claim 9, wherein the composition increases phosphorylcholine production of the glioblastoma cell.

14. The method of claim 9, wherein the glioblastoma cells are sensitized to an agent to which they have become refractory as a result of increased ceramide production in the glioblastoma cell.

15. The method of claim 9, wherein the glioblastoma cells are sensitized to an agent to which they have become refractory as a result of increased phosphorylcholine production in the glioblastoma cell.

16.-20. (canceled)


SEQUENCE LISTING (100 sequences)

SEQ ID NO: 1 - protein, 8 aa, Homo sapiens
GGTFSNYA

SEQ ID NO: 2 - protein, 8 aa, Homo sapiens
IIPIVSIA

SEQ ID NO: 3 - protein, 18 aa, Homo sapiens
ARSHYNDRSGYEQYYFDF

SEQ ID NO: 4 - protein, 125 aa, Homo sapiens
EVQLVQSGAEVKKPGSSVKVSCKASGGTFSNYAISWVRQAPGQGLEWMGRIIPIVSIANYAQEFQGRVTISADTSTRTAYMELSGLRSEDTAVYYCARSHYNDRSGYEQYYFDFWGQGTLVTVSS

SEQ ID NO: 5 - protein, 329 aa, Homo sapiens
STKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 6 aa, Homo sapiens
QSVSSY

SEQ ID NO: 7 - skipped

SEQ ID NO: 8 - protein, 9 aa, Homo sapiens
QQRSIRALA

SEQ ID NO: 9 - protein, 107 aa, Homo sapiens
EIVLTQSPATLSLSPGERATLSCRASQSVSSYLAWYQQKPGQAPRLLIYDASNRATGIPARFSGSGSGTDFALTISSLEPEDFAIYYCQQRSIRALAFGGGTKVEIK

SEQ ID NO: 10 - protein, 107 aa, Homo sapiens [sequence omitted]

SEQ ID NO: 11 - protein, 8 aa, Homo sapiens
GASIISYY

SEQ ID NO: 12 - protein, 7 aa, Homo sapiens
VYYSGST

SEQ ID NO: 13 - protein, 13 aa, Homo sapiens
ARDYGGNANYFGY

SEQ ID NO: 14 - protein, 119 aa, Homo sapiens
QVQLQESGPGLVKPSETLSLTCTVSGASIISYYWNWIRQTPGKGLEWIGNVYYSGSTNYNPSLKSRVTISVDTSKNQFSLKVSSVTAADTAVYYCARDYGGNANYFGYWGQGTLVTVSS

SEQ ID NO: 15 - protein, 329 aa, Homo sapiens; identical to SEQ ID NO: 5

SEQ ID NO: 16 - protein, 6 aa, Homo sapiens
QDIGKY

SEQ ID NO: 17 - skipped

SEQ ID NO: 18 - protein, 9 aa, Homo sapiens
QQYANLPLT

SEQ ID NO: 19 - protein, 107 aa, Homo sapiens
DIQMTQSPSSLSASVGDRVTITCQASQDIGKYLSWSQQKPGKAPNLLIYDASDLETGVPSRFSGSGSGTDFTFTISSLQPEDIATYYCQQYANLPLTFGGGTKVEIK

SEQ ID NO: 20 - protein, 107 aa, Homo sapiens [sequence omitted]

SEQ ID NO: 21 - DNA, 376 bp, Homo sapiens
  source 1..376: mol_type = genomic DNA
gaggtgcagctggtgcagtctggggctgaggtgaagaagcctgggtcctcggtgaaggtatcctgcaaggcttctggaggcaccttcagcaactatgctatcagctgggtgcgacaggccccgggacaagggcttgagtggatgggaaggatcatccccatcgttagtatagcaaactacgcacaggagtttcagggcagagtcacgattagcgcggacacatccacgcgcacagcctatatggaactcagcggcctgagatctgaggacacggccgtgtattactgtgcgaggtcgcattacaatgataggagtggttatgaacaatattactttgacttctggggccagggaaccctggtcaccgtctcctcag

SEQ ID NO: 22 - DNA, 322 bp, Homo sapiens
  source 1..322: mol_type = genomic DNA
gaaattgtgttgacacagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcgctctcaccatcagcagcctagagcctgaagactttgcaatttattactgtcagcaacgtagcatccgggcgctcgctttcggcggagggaccaaggtggaaatcaaac

SEQ ID NO: 23 - DNA, 358 bp, Homo sapiens
  source 1..358: mol_type = genomic DNA
caggtgcagctgcaggagtcgggcccaggactggtgaagccttcggagaccctgtccctcacctgcactgtatctggtgcctccatcattagttactactggaactggatccggcagaccccagggaagggactggagtggattgggaatgtctattacagtgggagcaccaactacaacccctccctcaagagtcgagtcaccatatcagtagacacgtccaagaaccagttctccctgaaggtgagctctgtgaccgctgcggacacggccgtctattactgtgcgagagactacggtggtaacgcgaactactttgggtactggggccagggaaccctggtcaccgtctcctcag

SEQ ID NO: 24 - DNA, 322 bp, Homo sapiens
  source 1..322: mol_type = genomic DNA
gacatccagatgacccagtctccatcctccctgtctgcatctgtaggagacagagtcaccatcacttgccaggcgagtcaggacattggcaagtatttaagttggtctcagcagaaaccagggaaagcccctaacctcctgatctacgatgcatccgatttggaaacaggggtcccatctaggttcagtggaagtggatctgggacagattttactttcaccatcagcagcctgcagcccgaagatattgcaacatattactgtcaacagtatgctaatctcccgctcactttcggcggagggaccaaggtggagatcaaac

SEQ ID NO: 25 - DNA, 18 bp, synthetic construct
  misc_feature 1..18: note = Synthetic: VH1/7_ext
  source 1..18: mol_type = other DNA
atggactggacctggags

SEQ ID NO: 26 - DNA, 22 bp, synthetic construct
  misc_feature 1..22: note = Synthetic: VH2_ext
  source 1..22: mol_type = other DNA
catactttgttccacgctcctg

SEQ ID NO: 27 - DNA, 20 bp, synthetic construct
  misc_feature 1..20: note = Synthetic: VH3_ext
  source 1..20: mol_type = other DNA
aggtgtccagtgtsargtgc

SEQ ID NO: 28 - DNA, 17 bp, synthetic construct
  misc_feature 1..17: note = Synthetic: VH4_ext
  source 1..17: mol_type = other DNA
gtggcrgctcccagatg

SEQ ID NO: 29 - DNA, 22 bp, synthetic construct
  misc_feature 1..22: note = Synthetic: VH5_ext
  source 1..22: mol_type = other DNA
gttctccaaggagtctgtkccg

SEQ ID NO: 30 - DNA, 23 bp, synthetic construct
  misc_feature 1..23: note = Synthetic: VH6_ext
  source 1..23: mol_type = other DNA
ctgtctccttcctcatcttcctg

SEQ ID NO: 31 - DNA, 21 bp, synthetic construct
  misc_feature 1..21: note = Synthetic: IgG_ext_rev
  source 1..21: mol_type = other DNA
tcttgtccaccttggtgttgc

SEQ ID NO: 32 - DNA, 22 bp, synthetic construct
  misc_feature 1..22: note = Synthetic: VK1_ext
  source 1..22: mol_type = other DNA
gagggtccyygctcagctcctg

SEQ ID NO: 33 - DNA, 22 bp, synthetic construct
  misc_feature 1..22: note = Synthetic: VK2_ext
  source 1..22: mol_type = other DNA
gaggctccytgctcagctyctg

SEQ ID NO: 34 - DNA, 24 bp, synthetic construct
  misc_feature 1..24: note = Synthetic: VK3_ext
  source 1..24: mol_type = other DNA
ctcttcctcctgctactctggctc

SEQ ID NO: 35 - DNA, 26 bp, synthetic construct
  misc_feature 1..26: note = Synthetic: VK4_ext
  source 1..26: mol_type = other DNA
gtgttgcagacccaggtcttcatttc

SEQ ID NO: 36 - DNA, 24 bp, synthetic construct
  misc_feature 1..24: note = Synthetic: VK5_ext
  source 1..24: mol_type = other DNA
gttcacctcctcagcttcctcctc

SEQ ID NO: 37 - DNA, 24 bp, synthetic construct
  misc_feature 1..24: note = Synthetic: 3 CK 543-566
  source 1..24: mol_type = other DNA
gtttctcgtagtctgctttgctca

SEQ ID NO: 38 - DNA, 17 bp, synthetic construct
  misc_feature 1..17: note = Synthetic: VL1/2_ext
  misc_feature 9: note = n is deoxyinosine
  source 1..17: mol_type = other DNA
grcacaggntcytgggc

SEQ ID NO: 39 - DNA, 17 bp, synthetic construct
  misc_feature 1..17: note = Synthetic: VL3a_ext
  misc_feature 10: note = n is deoxyinosine
  source 1..17: mol_type = other DNA
cagkctctgngrcctcc

SEQ ID NO: 40 - DNA, 17 bp, synthetic construct
  misc_feature 1..17: note = Synthetic: VL3b_ext
  misc_feature 5: note = n is deoxyinosine
  source 1..17: mol_type = other DNA
caggntcygtggcctcc

SEQ ID NO: 41 - DNA, 21 bp, synthetic construct
  misc_feature 1..21: note = Synthetic: VL4_ext
  source 1..21: mol_type = other DNA
ccactgcacagggtctctctc

SEQ ID NO: 42 - DNA, 22 bp, synthetic construct
  misc_feature 1..22: note = Synthetic: VL5_ext
  source 1..22: mol_type = other DNA
ctcactgcacaggttccctctc

SEQ ID NO: 43 - DNA, 18 bp, synthetic construct
  misc_feature 1..18: note = Synthetic: VL7_ext
  source 1..18: mol_type = other DNA
cacttgctgcccagggtc

SEQ ID NO: 44 - DNA, 24 bp, synthetic construct
  misc_feature 1..24: note = Synthetic: VL8_ext
  source 1..24: mol_type = other DNA
ctccttgcttatggrtcaggrgtg

SEQ ID NO: 45 - DNA, 15 bp, synthetic construct
  misc_feature 1..15: note = Synthetic: VL9/10/11_ext
  misc_feature 7: note = n is deoxyinosine
  source 1..15: mol_type = other DNA
ccagscngkgctgac

SEQ ID NO: 46 - DNA, 18 bp, synthetic construct
  misc_feature 1..18: note = Synthetic: CL_ext_rev
  source 1..18: mol_type = other DNA
gacggggctgcyatctgc

SEQ ID NO: 47 - DNA, 20 bp, synthetic construct
  misc_feature 1..20: note = Synthetic: VH1/5/7_nested
  misc_feature 6: note = n is deoxyinosine
  source 1..20: mol_type = other DNA
saggtncagctggtgcartc

SEQ ID NO: 48 - DNA, 25 bp, synthetic construct
  misc_feature 1..25: note = Synthetic: VH2_nested
  source 1..25: mol_type = other DNA
cagrtcaccttgarggagtctggtc

SEQ ID NO: 49 - DNA, 20 bp, synthetic construct
  misc_feature 1..20: note = Synthetic: VH3_nested_A
  source 1..20: mol_type = other DNA
saggtgcagctggtggagtc

SEQ ID NO: 50 - DNA, 20 bp, synthetic construct
  misc_feature 1..20: note = Synthetic: VH3_nested_B
  source 1..20: mol_type = other DNA
gargtgcagctgktggagtc

SEQ ID NO: 51 - DNA, 20 bp, synthetic construct
  misc_feature 1..20: note = Synthetic: VH4_nested_A
  source 1..20: mol_type = other DNA
cagstgcagctgcaggagtc

SEQ ID NO: 52 - DNA, 21 bp, synthetic construct
  misc_feature 1..21: note = Synthetic: VH4_nested_B
  source 1..21: mol_type = other DNA
caggtgcagctacagcagtgg

SEQ ID NO: 53 - DNA, 22 bp, synthetic construct
  misc_feature 1..22: note = Synthetic: VH6_nested
  source 1..22: mol_type = other DNA
caggtacagctgcagcagtcag

SEQ ID NO: 54 - DNA, 21 bp, synthetic construct
  misc_feature 1..21: note = Synthetic: Nested_rev
  source 1..21: mol_type = other DNA
gtccttgaccaggcagcccag

SEQ ID NO: 55 - DNA, 24 bp, synthetic construct
  misc_feature 1..24: note = Synthetic: VK1_nested
  source 1..24: mol_type = other DNA
catccagwtgacccagtctccatc

SEQ ID NO: 56 - DNA, 23 bp, synthetic construct
  misc_feature 1..23: note = Synthetic: VK2_nested
  source 1..23: mol_type = other DNA
ttgtgatgacycagwctccactc

SEQ ID NO: 57 - DNA, 18 bp, synthetic construct
  misc_feature 1..18: note = Synthetic: VK3_nested
  source 1..18: mol_type = other DNA
cagtctccagscaccctg

SEQ ID NO: 58 - DNA, 23 bp, synthetic construct
  misc_feature 1..23: note = Synthetic: VK4_nested
  source 1..23: mol_type = other DNA
catcgtgatgacccagtctccag

SEQ ID NO: 59 - DNA, 22 bp, synthetic construct
  misc_feature 1..22: note = Synthetic: VK5_nested
  source 1..22: mol_type = other DNA
gaaacgacactcacgcagtctc

SEQ ID NO: 60 - DNA, 22 bp, synthetic construct
  misc_feature 1..22: note = Synthetic: 3 CK 494-516
  source 1..22: mol_type = other DNA
gtgctgtccttgctgtcctgct

SEQ ID NO: 61 - DNA, 19 bp, synthetic construct
  misc_feature 1..19: note = Synthetic: VL1_int
  misc_feature 10: note = n is deoxyinosine
  source 1..19: mol_type = other DNA
cagtctgtsntgacgcagc

SEQ ID NO: 62 - DNA, 20 bp, synthetic construct
  misc_feature 1..20: note = Synthetic: VL2_int
  source 1..20: mol_type = other DNA
cagtctgccctgaytcagcc

SEQ ID NO: 63 - DNA, 22 bp, synthetic construct
  misc_feature 1..22: note = Synthetic: VL3_int
  source 1..22: mol_type = other DNA
cctcctatgwgctgacwcagcc

SEQ ID NO: 64 - DNA, 22 bp, synthetic construct
  misc_feature 1..22: note = Synthetic: VL4_int
  source 1..22: mol_type = other DNA
cccagcytgtgctgactcaatc

SEQ ID NO: 65 - DNA, 19 bp, synthetic construct
  misc_feature 1..19: note = Synthetic: VL5_int
  source 1..19: mol_type = other DNA
cagsctgtgctgactcagc

SEQ ID NO: 66 - DNA, 19 bp, synthetic construct
  misc_feature 1..19: note = Synthetic: VL7/8_int
  misc_feature 4: note = n is deoxyinosine
  source 1..19: mol_type = other DNA
cagnctgtggtgacycagg

SEQ ID NO: 67 - DNA, 18 bp, synthetic construct
  misc_feature 1..18: note = Synthetic: VL9/10/11_int
  source 1..18: mol_type = other DNA
gctgactcagccrccytc

SEQ ID NO: 68 - DNA, 22 bp, synthetic construct
  misc_feature 1..22: note = Synthetic: CL_int_rev
  source 1..22: mol_type = other DNA
cyagtgtggccttgttggcttg

SEQ ID NO: 69 - DNA, 30 bp, synthetic construct
  misc_feature 1..30: note = Synthetic: 3'RSRII_JK1/2/4
  source 1..30: mol_type = other DNA
gccacggtccgtttgatytccaccttggtc

SEQ ID NO: 70 - DNA, 30 bp, synthetic construct
  misc_feature 1..30: note = Synthetic: 3'RSRII_JK3
  source 1..30: mol_type = other DNA
gccacggtccgtttgatatccactttggtc

SEQ ID NO: 71 - DNA, 30 bp, synthetic construct
  misc_feature 1..30: note = Synthetic: 3'RSRII_JK5
  source 1..30: mol_type = other DNA
gccacggtccgtttaatctccagtcgtgtc

SEQ ID NO: 72 - DNA, 38 bp, synthetic construct
  misc_feature 1..38: note = Synthetic: 5' AgeI VH1/5/7
  source 1..38: mol_type = other DNA
ctgcaaccggtgtacattccgaggtgcagctggtgcag

SEQ ID NO: 73 - DNA, 38 bp, synthetic construct
  misc_feature 1..38: note = Synthetic: 5' AgeI VH3
  source 1..38: mol_type = other DNA
ctgcaaccggtgtacattctgaggtgcagctggtggag

SEQ ID NO: 74 - DNA, 38 bp, synthetic construct
  misc_feature 1..38: note = Synthetic: 5' AgeI VH323
  source 1..38: mol_type = other DNA
ctgcaaccggtgtacattctgaggtgcagctgttggag

SEQ ID NO: 75 - DNA, 38 bp, synthetic construct
  misc_feature 1..38: note = Synthetic: 5' AgeI VH4
  source 1..38: mol_type = other DNA
ctgcaaccggtgtacattcccaggtgcagctgcaggag

SEQ ID NO: 76 - DNA, 40 bp, synthetic construct
  misc_feature 1..40: note = Synthetic: 5' AgeI VH4-34
  source 1..40: mol_type = other DNA
ctgcaaccggtgtacattcccaggtgcagctacagcagtg

SEQ ID NO: 77 - DNA, 38 bp, synthetic construct
  misc_feature 1..38: note = Synthetic: 5' AgeI VH1-18
  source 1..38: mol_type = other DNA
ctgcaaccggtgtacattcccaggttcagctggtgcag

SEQ ID NO: 78 - DNA, 38 bp, synthetic construct
  misc_feature 1..38: note = Synthetic: 5' AgeI VH1-24
  source 1..38: mol_type = other DNA
ctgcaaccggtgtacattcccaggtccagctggtacag

SEQ ID NO: 79 - DNA, 38 bp, synthetic construct
  misc_feature 1..38: note = Synthetic: 5' AgeI VH3-9/30/33
  source 1..38: mol_type = other DNA
ctgcaaccggtgtacattctgaagtgcagctggtggag

SEQ ID NO: 80 - DNA, 38 bp, synthetic construct
  misc_feature 1..38: note = Synthetic: 5' AgeI VH6-1
  source 1..38: mol_type = other DNA
ctgcaaccggtgtacattcccaggtacagctgcagcag

SEQ ID NO: 81 - DNA, 40 bp, synthetic construct
  misc_feature 1..40: note = Synthetic: 5' AgeI VK1
  source 1..40: mol_type = other DNA
ctgcaaccggtgtacattctgacatccagatgacccagtc

SEQ ID NO: 82 - DNA, 46 bp, synthetic construct
  misc_feature 1..46: note = Synthetic: 5' AgeI VK1-9/1-13
  source 1..46: mol_type = other DNA
ttgtgctgcaaccggtgtacattcagacatccagttgacccagtct

SEQ ID NO: 83 - DNA, 40 bp, synthetic construct
  misc_feature 1..40: note = Synthetic: 5' AgeI VK1D-43/1-8
  source 1..40: mol_type = other DNA
ctgcaaccggtgtacattgtgccatccggatgacccagtc

SEQ ID NO: 84 - DNA, 40 bp, synthetic construct
  misc_feature 1..40: note = Synthetic: 5' AgeI VK2
  source 1..40: mol_type = other DNA
ctgcaaccggtgtacatggggatattgtgatgacccagac

SEQ ID NO: 85 - DNA, 40 bp, synthetic construct
  misc_feature 1..40: note = Synthetic: 5' AgeI VK2-28/2-30
  source 1..40: mol_type = other DNA
ctgcaaccggtgtacatggggatattgtgatgactcagtc

SEQ ID NO: 86 - DNA, 45 bp, synthetic construct
  misc_feature 1..45: note = Synthetic: 5' AgeI VK3-11/3D-11
  source 1..45: mol_type = other DNA
ttgtgctgcaaccggtgtacattcagaaattgtgttgacacagtc

SEQ ID NO: 87 - DNA, 40 bp, synthetic construct
  misc_feature 1..40: note = Synthetic: 5' AgeI VK3-15/3D-15
  source 1..40: mol_type = other DNA
ctgcaaccggtgtacattcagaaatagtgatgacgcagtc

SEQ ID NO: 88 - DNA, 46 bp, synthetic construct
  misc_feature 1..46: note = Synthetic: 5' AgeI VK320/3D-20
  source 1..46: mol_type = other DNA
ttgtgctgcaaccggtgtacattcagaaattgtgttgacgcagtct

SEQ ID NO: 89 - DNA, 40 bp, synthetic construct
  misc_feature 1..40: note = Synthetic: 5' AgeI VK4-1
  source 1..40: mol_type = other DNA
ctgcaaccggtgtacattcggacatcgtgatgacccagtc

SEQ ID NO: 90 - DNA, 38 bp, synthetic construct
  misc_feature 1..38: note = Synthetic: 5' AgeI VL1
  source 1..38: mol_type = other DNA
ctgctaccggttcctgggcccagtctgtgctgackcag

SEQ ID NO: 91 - DNA, 38 bp, synthetic construct
  misc_feature 1..38: note = Synthetic: 5' AgeI VL2
  source 1..38: mol_type = other DNA
ctgctaccggttcctgggcccagtctgccctgactcag

SEQ ID NO: 92 - DNA, 38 bp, synthetic construct
  misc_feature 1..38: note = Synthetic: 5' AgeI VL3
  source 1..38: mol_type = other DNA
ctgctaccggttctgtgacctcctatgagctgacwcag

SEQ ID NO: 93 - DNA, 37 bp, synthetic construct
  misc_feature 1..37: note = Synthetic: 5' AgeI VL4/5
  source 1..37: mol_type = other DNA
ctgctaccggttctctctcscagcytgtgctgactca

SEQ ID NO: 94 - DNA, 38 bp, synthetic construct
  misc_feature 1..38: note = Synthetic: 5' AgeI VL6
  source 1..38: mol_type = other DNA
ctgctaccggttcttgggccaattttatgctgactcag

SEQ ID NO: 95 - DNA, 38 bp, synthetic construct
  misc_feature 1..38: note = Synthetic: 5' AgeI VL7/8
  source 1..38: mol_type = other DNA
ctgctaccggttccaattcycagrctgtggtgacycag

SEQ ID NO: 96 - DNA, 32 bp, synthetic construct
  misc_feature 1..32: note = Synthetic: 3' SalI JH1/2/4/5
  source 1..32: mol_type = other DNA
tgcgaagtcgacgctgaggagacggtgaccag

SEQ ID NO: 97 - DNA, 34 bp, synthetic construct
  misc_feature 1..34: note = Synthetic: 3' SalI JH3
  source 1..34: mol_type = other DNA
tgcgaagtcgacgctgaagagacggtgaccattg

SEQ ID NO: 98 - DNA, 33 bp, synthetic construct
  misc_feature 1..33: note = Synthetic: 3' SalI JH6
  source 1..33: mol_type = other DNA
tgcgaagtcgacgctgaggagacggtgaccgtg

SEQ ID NO: 99 - DNA, 28 bp, synthetic construct
  misc_feature 1..28: note = Synthetic: 3' XhoI C1
  source 1..28: mol_type = other DNA
ctcctcactcgagggygggaacagagtg

SEQ ID NO: 100 - DNA, 21 bp, synthetic construct
  misc_feature 1..21: note = Synthetic: AbVec
  source 1..21: mol_type = other DNA
gcttcgttagaacgcggctac